(19) United States
(12) Patent Application Publication
(10) Pub. No.: US 2005/0131028 A1
(43) Pub. Date: Jun. 16, 2005

(54) Methods and compositions for the extended duration treatment of pain, inflammation and inflammation-related disorders

(75) Inventors: Jeffery S. Carter, Chesterfield, MO (US); Bernard D. Hummel, Kalamazoo, MI (US)

Correspondence Address: Charles E. Dunlap;Nelson Mullins Riley & Scarborough, LLP, 17th Floor, 1320 Main Street, Columbia, SC, 29201, US

(73) Assignee: Pharmacia Corporation, Chesterfield, MO (US)

(21) Appl. No.: 10/936,875

(22) Filed: Sep. 9, 2004

Related U.S. Application Data
(60) Provisional application No. 60/502,053, filed on Sep. 11, 2003.

(51) Int. Cl. A61K 31/4439; A61K 31/44; A61K 31/4152; A61K 31/365

(52) U.S. Cl. 514/341; 514/357; 514/406; 514/471

(57) ABSTRACT

A method is disclosed for providing extended duration treatment or prevention of pain, inflammation and inflammation-related disorders in a subject in need of such extended duration treatment or prevention by administering to the subject a Cox-2 selective inhibitor having certain added substituent groups. Also disclosed is a method for extending the duration of the plasma half-life of diaryl-substituted Cox-2 selective inhibitors by substituting halogen, haloalkyl, haloalkoxy, alkyl, nitrile, cyano or alkoxy groups at certain positions on the diaryl-substituted rings of the inhibitor molecule. A novel composition comprising such extended duration Cox-2 selective inhibitors is also disclosed.

### CROSS-REFERENCE TO RELATED APPLICATIONS

This application is related to and claims the priority benefit of U.S. Provisional Patent Application Ser. No. 60/502,053 filed Sep. 11, 2003, which is incorporated by reference herein in its entirety.

### BACKGROUND OF THE INVENTION

(1) Field of the Invention

The present invention relates generally to extended duration Cox-2 selective inhibitors useful for the prevention and treatment of pain, inflammation, and inflammation-related disorders, and, more particularly, to a method of increasing the plasma half-life of Cox-2 selective inhibitors and a method for their use.

(2) Description of Related Art

Extended duration pharmaceutical formulations provide several advantages to physicians and their patients over single-dosage rapid release formulations. Extended duration formulations provide a longer period of pharmacological response after the administration of the drug than is experienced after the administration of the rapid release forms (e.g. daily dosing). A longer period of pharmacological response can be beneficial for a patient in terms of cost effectiveness, ease of dosing, patient compliance, safety, and consistency of therapeutic response. This is particularly important for pain and inflammation treatments that must be maintained over long intervals at therapeutically effective levels to effectuate steady-state blood plasma levels of a therapeutic drug.

Historically, physicians have treated inflammation with a regimen of nonsteroidal anti-inflammatory drugs (NSAIDS) such as aspirin, naproxen and ibuprofen. Undesirably, however, some NSAIDS are known to cause gastrointestinal bleeding or ulcers in patients undergoing long-term regimens of NSAID therapy. In the USA, the widespread use of NSAIDS now accounts for up to 107,000 hospitalizations and 16,500 deaths annually. The annual health care cost of these hospitalizations has been estimated to be over $2 billion. American College of Rheumatology Ad Hoc Committee on Clinical Guidelines: Guidelines for the management of rheumatoid arthritis. Arthritis & Rheumatism 39:713-722 (1996).

A reduction of unwanted side effects of common NSAIDS was made possible by the discovery that two cyclooxygenases are involved in the transformation of arachidonic acid as the first step in the prostaglandin synthesis pathway. These enzymes have been termed cyclooxygenase-1 (Cox-1) and cyclooxygenase-2 (Cox-2). See Needleman, P. et al., J. Rheumatol., 24, Suppl.49:6-8 (1997).

Cyclooxygenase enzymes (Cox; prostaglandin synthase, EC 1.14.99.1) are responsible for metabolizing arachidonic acid to prostaglandin H₂, which, in turn, serves as a precursor for the biosynthesis of several other prostaglandins, thromboxanes, and prostacyclin. See e.g., Hamberg, et al., Proc. Natl. Acad. Sci. USA 70:899-903 (1973). Cyclooxygenase exists in two forms. Cox-1 is a constitutive enzyme responsible for the biosynthesis of prostaglandins in the gastric mucosa and in the kidney. Cox-2 is an enzyme that is produced by an inducible gene that is responsible for the biosynthesis of prostaglandins in inflammatory cells. Inflammation causes the induction of Cox-2, leading to the release of prostanoids (prostaglandin E2), which sensitize peripheral nociceptor terminals and produce localized pain hypersensitivity, inflammation, and oedema. See e.g., Samad, T. A. et al., Nature, 410(6827):471-5 (2001). Many common NSAIDs are now known to be inhibitors of both Cox-1 and Cox-2. Accordingly, when administered in sufficiently high levels, these NSAIDs not only alleviate the inflammatory consequences of Cox-2 activity, but also inhibit the beneficial gastric maintenance activities of Cox-1.

Research into the area of arachidonic acid metabolism has resulted in the discovery of compounds that selectively inhibit the Cox-2 enzyme to a greater extent than the activity of Cox-1. The Cox-2-selective inhibitors are believed to offer advantages that include the capacity to prevent or reduce pain and inflammation while avoiding harmful side effects associated with the inhibition of Cox-1. Thus, Cox-2 selective inhibitors have shown great promise for use in therapies—especially in therapies that require maintenance administration, such as for pain and inflammation control for arthritis.

With some Cox-2 selective inhibitors, in particular, those that are diaryl-substituted compounds, it is typical to employ a daily dosing regimen. The requirement for daily dosing arises from the fact that for most Cox-2 selective inhibitors of this class, the drug is metabolized and/or excreted to sub-therapeutic plasma levels within a day of administration. Thus, a patient re-doses, at the minimum, every day to maintain therapeutic plasma levels of the drug.

It would be advantageous, in some instances, to provide methods and compositions that provide a longer period of pharmacological response for the treatment or prevention of inflammation. Also needed are methods for increasing the plasma half-life of standard pharmaceutical compositions useful for treating inflammation. Extended duration pharmaceutical compositions would improve the cost effectiveness, ease of dosing, patient compliance, safety, and consistency of therapeutic response for treating or preventing inflammation.

It would be useful; therefore, if a Cox-2 selective inhibitor composition having an increased plasma half-life could be provided. It would also be useful to provide a method of increasing the plasma half-life of a Cox-2 selective inhibitor. It would also be useful to provide a method of treating inflammation by administrating to a patient in need thereof, an extended duration Cox-2 selective inhibitor that would require a less frequent dosing regimen than presently available Cox-2 inhibitors. Also useful would be to provide a method for providing extended duration Cox-2 selective inhibitors to reduce fluctuations in the plasma concentration of these drugs, especially in maintenance administrations.

### SUMMARY OF THE INVENTION

Briefly, therefore, the present invention is directed to a novel method of providing extended duration treatment or prevention of pain, inflammation and inflammation-related disorders in a subject in need of such extended duration treatment or prevention, the method comprising administering to the subject a compound having the formula: 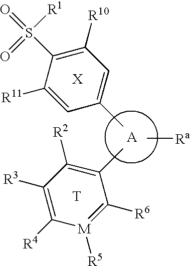
wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen; and
including the diastereomers, enantiomers, racemates, tautomers, salts, esters, amides and prodrugs thereof.

The present invention is also directed to a novel method of increasing the plasma half-life of a Cox-2 selective inhibitor comprising adding one or more substituent groups onto either or both of the “T” ring and “X” ring of a Cox-2 selective inhibitor having the structure: 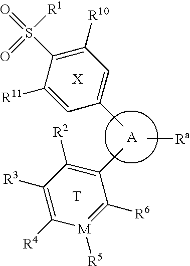
to provide an extended duration Cox-2 selective inhibitor wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen; and
including the diastereomers, enantiomers, racemates, tautomers, salts, esters, amides and prodrugs thereof.

Also provided is a novel method of reducing the dosing frequency of a diaryl-substituted Cox-2 selective inhibitor compound comprising:
a. adding one or more substituent groups onto either or both of the “T” ring and “X” ring of a Cox-2 selective inhibitor having the structure: 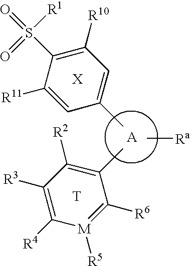
   to provide an extended duration Cox-2 selective inhibitor wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen; and
including the diastereomers, enantiomers, racemates, tautomers, salts, esters, amides and prodrugs thereof; and
b. administering a therapeutic amount of the resulting compound to a subject in need of such reduced frequency dosing.

Also provided is a therapeutic composition comprising a compound having the structure: 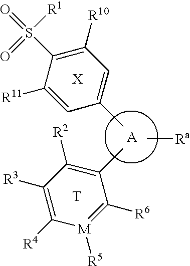
wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen; and
including the diastereomers, enantiomers, racemates, tautomers, salts, esters, amides and prodrugs thereof.

Among the several advantages found to be achieved by the present invention, therefore, may be noted the provision of a method for increasing the plasma half-life of a diaryl-substituted Cox-2 inhibitor compound, the provision of an extended duration diaryl-substituted Cox-2 inhibitor compound, the provision of a method for treating inflammation, comprising administering to a patient an extended duration Cox-2 selective inhibitor, the provision of a novel extended duration therapeutic composition, and the provision of a novel method of reducing the dosing frequency of a diaryl-substituted Cox-2 selective inhibitor compound.

### BRIEF DESCRIPTION OF THE DRAWINGS

In the Figures, the following letters are used to designate the corresponding chemical names:
A. 4-[5-(2,4-[difluorophenyl)-3-(trifluoromethyl)-1H-pyrazol-1-yl]benzenesulfonamide,
B. 4-[5-(4-chlorophenyl)-3-(trifluoromethyl)-1H-pyrazol-1-yl]benzenesulfonamide,
C. 4-[5-(2,4-[dichlorophenyl)-3-(trifluoromethyl)-1H-pyrazol-1-yl]benzenesulfonamide,
D. 4-[5-phenyl-3-(trifluoromethyl)-1H-pyrazol-1-yl]benzenesulfonamide,
E. 4-[5-(3,4-dichlorophenyl)-3-(trifluoromethyl)-1H-pyrazol-1-yl]benzenesulfonamide,
F. 4-[5-(4-fluorophenyl)-3-(trifluoromethyl)-1H-pyrazol-1-yl]benzenesulfonamide,
G. 4-[5-(3,4-difluorophenyl)-3-(trifluoromethyl)-1H-pyrazol-1-yl]benzenesulfonamide,
H. 4-[5-(4-methoxyphenyl)-3-(trifluoromethyl)-1H-pyrazol-1-yl]benzenesulfonamide,
I. 4-[5-(3-chloro-4-methoxyphenyl)-3-(trifluoromethyl)-1H-pyrazol-1-yl]benzenesulfonamide,
J. 4-[5-(3,5-dichloro-4-methoxyphenyl)-3-(trifluoromethyl)-1H-pyrazol-1-yl]benzenesulfonamide,
K. 4-[5-(4-methoxy-3-methylphenyl)-3-(trifluoromethyl)-1H-pyrazol-1-yl]benzenesulfonamide,
L. 4-[5-(4-methoxy-3,5-dimethylphenyl)-3-(trifluoromethyl)-1H-pyrazol-1-yl]benzenesulfonamide,
M. 4-[5-(3-chloro-4-methylphenyl)-3-(trifluoromethyl)-1H-pyrazol-1-yl]benzenesulfonamide,
N. 4-[5-(3,5-dichloro-4-methylphenyl)-3-(trifluoromethyl)-1H-pyrazol-1-yl]benzenesulfonamide,
O. 4-[5-(4-chloro-3-methylphenyl)-3-(trifluoromethyl)-1H-pyrazol-1-yl]benzenesulfonamide,
P. 4-[5-(4-chloro-3-methoxyphenyl)-3-(trifluoromethyl)-1H-pyrazol-1-yl]benzenesulfonamide,
Q. 4-[5-(4-bromo-3-methoxyphenyl)-3-(trifluoromethyl)-1H-pyrazol-1-yl]benzenesulfonamide, and
R. 4-[3-(4-chlorophenyl)-5-(trifluoromethyl)isoxazol-4-yl]benzenesulfonamide.

FIG. 1 is a graph of the plasma concentration in rats of a Cox-2 selective inhibitor as a function of time, along with the plasma concentration of a number of Cox-2 selective inhibitors having extended duration properties according to the present invention, where all Cox-2 inhibitors were orally administered once at 2 mg/kg.;

FIG. 2 is a graph of the paw volume in rats that were treated with an adjuvant to induce an arthritis-like condition and then with a number of Cox-2 selective inhibitors having extended duration properties according to the present invention as a function of time, where all Cox-2 inhibitors were orally administered once at 3 mg/kg;

FIG. 3 is a graph of a canine cassette-dosed pharmacokinetic study, where the mean plasma concentration in canines of a Cox-2 selective inhibitor (compound D identified above) is depicted as a function of time, along with the plasma concentration of a number of Cox-2 selective inhibitors having extended duration properties according to the present invention, where all Cox-2 selective inhibitors were orally administered once at 2 mg/kg;

FIG. 4 is a graph of a canine pharmacokinetic summary, where the mean plasma concentration in canines of a Cox-2 selective inhibitor (D) is depicted as a function of time, along with the plasma concentration of a number of Cox-2 selective inhibitors having extended duration properties according to the present invention, where all Cox-2 inhibitors were orally administered once at 2 mg/kg or 4 mg/kg;

FIG. 5 is a graph of a canine acute pain efficacy model showing the mean lameness in canines administered a Cox-2 selective inhibitor (D) plotted as a function of time, along with a marketed nonsteroidal anti-inflammatory drug for canines (Rimadyl®), where the Cox-2 inhibitor was orally administered once at 2.0 mg/kg and Rimadyl® was orally administered at 2.2 mg/kg twice daily for 4 days. Note that efficacy of a single 2 mg/kg oral dose of 4-[5-phenyl-3-(trifluoromethyl)-1H-pyrazol-1-yl]benzenesulfonamide lasts between 12 and 24 hrs, and would need to be administered approximately once daily to maintain therapeutic effectiveness. Rimadyl® was measured ˜7 hours following the morning dose on the 4^{th} consecutive day of dosing;

FIG. 6 is a graph of a canine acute pain efficacy model showing the mean lameness in canines administered a Cox-2 selective inhibitor (F-(4-[5-(4-fluorophenyl)-3-(trifluoromethyl)-1H-pyrazol-1yl]benzenesulfonamide) having extended duration properties according to the present invention, plotted as a function of time, along with a marketed nonsteroidal anti-inflammatory drug for canines (Rimadyl®), where the extended duration Cox-2 inhibitor was orally administered once at 2.0 mg/kg and Rimadyl® was orally administered at 2.2 mg/kg twice daily for 4 days. Note that efficacy of a single 2 mg/kg oral dose of 4-[5-(4-fluorophenyl)-3-(trifluoromethyl)-1H-pyrazol-1-yl]benzenesulfonamide lasts for 7-28 days. Rimadyl® was measured ˜7 hours following the morning dose on the 4^{th} consecutive day of dosing; and

FIG. 7 is a graph of a canine acute pain efficacy model showing the mean lameness (expressed as a percent of control group) in canines administered the Cox-2 selective inhibitors (A-4-[5-(2,4-[difluorophenyl)-3-(trifluoromethyl)-1H-pyrazol-1-yl]benzenesulfonamide, C-4-[5-(2,4-[dichlorophenyl)-3-(trifluoromethyl)-1H-pyrazol-1-yl]benzenesulfonamide or E-4-[5-(3,4-dichlorophenyl)-3-(trifluoromethyl)-1H-pyrazol-1-yl]benzenesulfonamide) having extended duration properties according to the present invention plotted as a function of time, along with a marketed nonsteroidal anti-inflammatory drug for canines (Rimadyl®), where the extended duration Cox-2 inhibitors were orally administered once at 2.0 mg/kg (A) or 4 mg/kg (C and E) and Rimdayl® was orally administered at 2.2 mg/kg twice daily for 4 days. Note that efficacy of A, C, and E last for 14-28 days. Rimadyl® was measured at around 9 hours following the morning dose on the 4^{th} consecutive day of dosing.

### DETAILED DESCRIPTION OF THE PREFERRED EMBODIMENTS

In accordance with the present invention, it has been discovered that the in vivo plasma concentration of several commercially available diaryl-substituted Cox-2 selective inhibitors, including, but not limited to, diaryl-substituted pyrazolyl, diaryl-substituted pyridyl, diaryl-substituted imidazolyl, diaryl-substituted thiophenyl, diaryl-substituted isoxazolyl, diaryl-substituted oxazolyl and diaryl-substituted furanonyl Cox-2 selective inhibitors, can be extended significantly by the addition of certain substituent groups to the inhibitor molecule at specific locations. In fact, modification of some commercially available Cox-2 selective inhibitors according to the present method extends the duration of therapeutic drug levels as much as 600-fold over that provided by the unsubstituted drug—with no increase in dosage. Compared with the daily dosage regimen that is required for commercially available Cox-2 selective inhibitors, some of the compounds substituted according to the formulas described herein, demonstrate therapeutic levels for up to 49 days after only one dosage. Furthermore, in preferred embodiments, the selectivity of these extended duration Cox-2 selective inhibitors can be similar to, or even higher than, that of other commercially available Cox-2 selective inhibitors.

In particular, it has been discovered that certain diaryl-substituted Cox-2 selective inhibitors can be substituted with halogen, haloalkyl, haloalkoxy, alkyl, nitrile, cyano and alkoxy groups at certain locations to provide extended duration Cox-2 selective inhibitors that demonstrate a maximum plasma concentration equivalent to an immediate release oral medicament, yet maintain a therapeutically effective plasma concentration for a duration of greater than 12 hours without the need for an increase in dosage strength or for additional dosing.

An advantage of the extended duration Cox-2 inhibitors of the present invention is that less frequent dosing than is required for some commercially available Cox-2 selective inhibitors could be a convenience to the patient and could improve compliance with a prescribed dosing regimen. Another advantage of the use of the novel extended duration Cox-2 selective inhibitors is a lower variability in plasma levels, which can reduce fluctuations in the plasma concentration of these drugs, especially in maintenance administrations. Also, lower fluctuations in plasma concentrations may result in a patient receiving more consistent amounts of the drug in conservative or aggressive dosing regimens. See, e.g., U.S. Pat. No. 6,419,953 B1 to Qiu, et al. In addition, there are situations where some patients who are forgetful or otherwise incapacitated are only visited by health care personnel at widespread intervals. Thus, the availability of an extended duration Cox-2 selective inhibitor could lead to more dependable treatment for these patients.

As used herein, the terms “Cox-2 selective inhibitor”, or “Cox-2 selective inhibitor”, which can be used interchangeably herein, embrace compounds that selectively inhibit Cox-2 over Cox-1, and include pharmaceutically acceptable salts and prodrugs of those compounds.

As used herein, “halo” or “halogen” means fluorine, chlorine, bromine, iodine, or astatine.

As used herein, “haloalkyl” embraces radicals wherein any one or more of alkyl carbon atoms is substituted with a halo as defined above. For example, the term “haloalkyl” means a compound having fluorine, chlorine, bromine, iodine, or astatine covalently coupled with an alkyl, alkenyl, alkynyl, alkoxy, aralkyl, aryl, carbonyl, cycloalkyl, benzyl, phenyl, alicyclic or heterocyclic group.

As used herein, the term “cyano” refers to a functional group that consists of a carbon atom joined to a nitrogen atom by a triple bond; it can be joined to an atom or another group by a single bond to the carbon atom. When it is joined to an alkyl group or aryl group, it forms a “nitrile.”

As used herein, the terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals.

As used herein, the “alkoxy” or “alkoxyalkyl” radicals may be further substituted with one or more halo atoms, such as fluoro, chloro or bromo to provide “haloalkoxy” or “haloalkoxyalkyl” radicals. Examples of “alkoxy” radicals include methoxy, butoxy and trifluoromethoxy.

As used herein, the term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronapthyl, indane and biphenyl. As used herein, the term “diaryl” refers to two aryl groups.

As used herein, the term “heterocycle” means a saturated or unsaturated mono- or multi-ring carbocycle wherein one or more carbon atoms is replaced by one or more of N, S, P, or O. This includes, for example, structures such as: 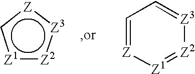
where Z, Z¹, Z², or Z³ is C, S, P, O, or N, with the proviso that one of Z, Z¹, Z², or Z³ is other than carbon. As used herein, and known to one of ordinary skill in the art, the use of a circular designation within a 5-membered heterocycle ring is meant to encompass the appropriate double bonding character between the ring atoms, regardless of whether the ring atoms are C, S, P, O, or N. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heterocyclic” embraces saturated, partially saturated and unsaturated heteroatom-containing ring-shaped radicals, where the heteroatoms may be selected from nitrogen, sulfur and oxygen. Examples of saturated heterocyclic radicals include pyrrolidyl and morpholinyl. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl and tetrazolyl. The term also embraces radicals where heterocyclic radicals are fused with aryl radicals. Examples of such fused bicyclic radicals include benzofuran, benzothiophene, and the like.

As used herein, the term “heteroaryl” means a fully unsaturated heterocycle, which can include, but is not limited to, furyl, thiophenyl, pyrryl, imidazolyl, pyrazolyl, pyridyl, thiazolyl, quinolinyl, isoquinolinyl, benzothienyl, and indolyl.

The present invention provides a method for increasing the effective half-life of diaryl-substituted Cox-2 selective inhibitors by the addition of one or more halogen, haloalkyl, haloalkoxy, alkyl, nitrile, cyano or alkoxy substituent groups covalently bonded to carbons within the “T” ring (ring “T” in formulas I, II, and III) or “X” ring (ring “X” in formulas I, II, and III). The present invention also provides a method for using halogen, haloalkyl, haloalkoxy, alkyl, nitrile, cyano or alkoxy additions to extend the half-life of diaryl-substituted Cox-2 selective inhibitors for use as an extended duration therapy for the treatment of inflammation.

The present invention further provides a method for increasing the bioavailability and plasma half-life of diaryl-substituted Cox-2 selective inhibitors in a subject by administering one or more of the halogen, haloalkyl, haloalkoxy, alkyl, nitrile, cyano or alkoxy substituted compounds described herein.

The present invention also comprises a therapeutic method for the extended duration treatment or prevention of pain, inflammation or inflammation-associated disorders in a subject in need of such extended duration treatment, the method comprising administering to the subject an effective amount of a compound of formulas I, II, and III, as described below.

As used herein, the term “subject” includes all vertebrates. The subject is typically an animal, and yet more typically is a mammal. “Mammal”, as that term is used herein, refers to any animal classified as a mammal, including humans, domestic and farm animals, livestock animals, companion animals, zoo, sports, or pet animals, such as dogs, horses, cats, cattle, etc.

The term “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

As used herein, the term “companion animals” refers to horses, cats and dogs. As used herein, the term “dog(s)” denotes any member of the species Canis familiaris, of which there are a large number of different breeds. While laboratory determinations of biological activity may have been carried out using a particular breed, it is contemplated that the inhibitory compounds of the present invention will be found to be useful for treating pain and inflammation in any of these numerous breeds. Dogs represent a particularly preferred class of patients in that they are well known as being very susceptible to chronic inflammatory processes such as osteoarthritis and degenerative joint disease, which in dogs often results from a variety of developmental diseases, e.g., hip dysplasia and osteochondrosis, as well as from traumatic injuries to joints.

As used herein, the terms “subject in need of” refer to any subject who is suffering from, or is predisposed to, an inflammation disorder or any inflammation-related complication described herein. For purposes of treatment a “subject in need of” includes any subject who is in need of the treatment of an inflammation-related disorder, or who needs treatment of an inflammation-related complication. For purposes of prevention, the subject is any subject, and preferably is a subject that is at risk for, or is predisposed to, developing an inflammation-related disorder or an inflammation-related complication.

As used herein, a subject that is “predisposed to” or “at risk for,” both of which are used interchangeably herein, includes any subject at risk for developing an inflammation-related disorder or any inflammation-related complication. The subject may be at risk due to genetic predisposition, diet, age, exposure to trauma, exposure to a potentially traumatic environment, exposure to an inflammation disorder-causing agent, and the like. The subject may also be at risk due to physiological factors such as anatomical and biochemical abnormalities and certain autoimmune diseases. For example, a subject diagnosed with rheumatoid arthritis, may be expected to be at risk for developing arthritis-related pain and inflammation. Likewise, canines (dogs) that are diagnosed with hip dysplasia are at risk for or predisposed to developing dehibilitating pain and inflammation.

As used herein, the terms “subject in need of extended duration treatment” includes subjects that require a lower frequency of dosing than is effectively possible by using currently commercially available Cox-2 selective inhibitors. Such a subject may require a lower frequency of dosing due to a difficulty with compliance at the regular dosing frequency. For example, a subject who regularly takes several pills per day or once daily or receives several injections per day or once daily, is less likely to successfully comply such a dosing regimen than a subject who would only need to take the pills or receive the injections once a week.

In addition, the compositions of the present invention are expected to reduce the fluctuations in plasma concentration that results from frequent dosing. Therefore, a “subject in need of extended duration treatment” includes subjects that would benefit from a reduction in the fluctuations of the plasma concentration of a Cox-2 selective inhibitor drug, especially in maintenance administrations. In still other embodiments, a “subject in need of extended duration treatment” includes subjects that require an improved consistency of therapeutic response for treating or preventing inflammation.

In other embodiments, a “subject in need of extended duration treatment” includes subjects that require an improvement in the cost effectiveness of taking a medication on a frequent dosing schedule, or that requires an improvement in safety by taking fewer dosages of a Cox-2 selective inhibitor.

The methods and compositions of the present invention would be useful for the treatment of inflammation in a subject with the compounds of Formula I, II, or III and for treatment of other inflammation-associated disorders, such as an analgesic in the treatment of pain and headaches, or as an antipyretic for the treatment of fever. As used herein, the terms “treating”, “treated”, or “to treat,” mean to alleviate symptoms, eliminate the causation either on a temporary or permanent basis, or to prevent or slow the appearance of symptoms. The term “treatment” includes alleviation, elimination of causation of or prevention of pain and/or inflammation associated with, but not limited to, any of the diseases or disorders described below.

In preferred embodiments, the methods and compositions of the present invention encompass the prevention and treatment of pain, inflammation and inflammation-related disorders.

In other preferred embodiments, the methods and compositions of the present invention encompass the prevention or treatment of several disorders selected from the group consisting of connective tissue and joint disorders, neoplasia disorders, cardiovascular disorders, otic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

As used herein, the terms “neoplasia” and “neoplasia disorder”, used interchangeably herein, refer to new cell growth that results from a loss of responsiveness to normal growth controls, e.g. to “neoplastic” cell growth. Neoplasia is also used interchangeably herein with the term “cancer” and for purposes of the present invention; cancer is one subtype of neoplasia. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth.

Both of the terms, “neoplasia” and “neoplasia disorder”, refer to a “neoplasm” or tumor, which may be benign, premalignant, metastatic, or malignant. Also encompassed by the present invention are benign, premalignant, metastatic, or malignant neoplasias. Also encompassed by the present invention are benign, premalignant, metastatic, or malignant tumors. Thus, all of benign, premalignant, metastatic, or malignant neoplasia or tumors are encompassed by the present invention and may be referred to interchangeably, as neoplasia, neoplasms or neoplasia-related disorders. Tumors are generally known in the art to be a mass of neoplasia or “neoplastic” cells. Although, it is to be understood that even one neoplastic cell is considered, for purposes of the present invention to be a neoplasm or alternatively, neoplasia.

In still other preferred embodiments, the methods and compositions of the present invention encompass the prevention and treatment of the connective tissue and joint disorders selected from the group consisting of arthritis, rheumatoid arthritis, spondyloarthopathies, gouty arthritis, carpal tunnel syndrome, canine hip dysplasia, systemic lupus erythematosus, osteoarthritis, tendonitis and bursitis.

In other preferred embodiments, the methods and compositions of the present invention encompass the prevention and treatment of the neoplasia disorders selected from the group consisting of acral lentiginous melanoma, actinic keratoses, adenocarcinoma, adenoid cycstic carcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cholangiocarcinoma, chondosarcoma, choriod plexus papilloma/carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

In other preferred embodiments, the methods and compositions of the present invention encompass the prevention and treatment of the cardiovascular disorders selected from the group consisting of myocardial ischemia, hypertension, hypotension, heart arrhythmias, pulmonary hypertension, hypokalemia, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis arteriosclerosis, atherosclerosis, calcification (such as vascular calcification and valvar calcification), coronary artery disease, heart failure, congestive heart failure, shock, arrhythmia, left ventricular hypertrophy, angina, diabetic nephropathy, kidney failure, eye damage, cardiac damage, diabetic cardiac myopathy, renal insufficiency, renal injury, renal arteriopathy, peripheral vascular disease, left ventricular hypertrophy, cognitive dysfunction, stroke, and headache.

In other preferred embodiments, the methods and compositions of the present invention encompass the prevention and treatment of the metabolic disorders selected from the group consisting of obesity, overweight, type I and type II diabetes, hypothyroidism, and hyperthyroidism.

In other preferred embodiments, the methods and compositions of the present invention encompass the prevention and treatment of the respiratory disorders selected from the group consisting of asthma, bronchitis, chronic obstructive pulmonary disease (COPD), cystic fibrosis, pulmonary embolism, pneumonia, pulmonary fibrosis, respiratory failure, acute respiratory distress syndrome and emphysema.

In other preferred embodiments, the methods and compositions of the present invention encompass the prevention and treatment of the angiogenesis-related disorders selected from the group consisting of angiofibroma, neovascular glaucoma, arteriovenous malformations, arthritis, osler-weber syndrome, atherosclerotic plaques, psoriasis, corneal graft neovascularization, pyogenic granuloma, delayed wound healing, retrolental fibroplasias, diabetic retinopathy, scleroderma, granulations, solid tumors, hemangioma, trachoma, hemophilic joints, vascular adhesions, hypertrophic scars, age-related macular degeneration, coronary artery disease, stroke, cancer, AIDS complications, ulcers and infertility.

In other preferred embodiments, the methods and compositions of the present invention encompass the prevention and treatment of the infectious diseases and disorders selected from the group consisting of viral infections, bacterial infections, prion infections, spirochetes infections, mycobacterial infections, rickettsial infections, chlamydial infections, parasitic infections and fungal infections.

In still further embodiments, the methods and compositions of the present invention encompass the prevention and treatment of the infectious diseases and disorders selected from the group consisting of hepatitis, HIV (AIDS), small pox, chicken pox, common cold, influenza, warts, oral herpes, genital herpes, herpes zoster, bovine spongiform encephalopathy, septicemia, streptococcus infections, staphylococcus infections, anthrax, severe acquired respiratory syndrome (SARS), malaria, African sleeping sickness, yellow fever, chlamydia, botulism, canine heartworm, rocky mountain spotted fever, lyme disease, cholera, syphilis, gonorrhea, encephalitis, pneumonia, conjunctivitis, yeast infections, rabies, dengue fever, Ebola, measles, mumps, rubella, West Nile virus, meningitis, gastroenteritis, tuberculosis, hepatitis, and scarlet fever.

In other preferred embodiments, the methods and compositions of the present invention encompass the prevention and treatment of the neurological and neurodegenerative disorders selected from the group consisting of headaches, migraine headaches, Alzheimer's disease, Parkinson's disease, dementia, memory loss, senility, amyotrophy, ALS, amnesia, seizures, multiple sclerosis, muscular dystrophies, epilepsy, schizophrenia, depression, anxiety, attention deficit disorder, hyperactivity, bulimia, anorexia nervosa, anxiety, autism, phobias, spongiform encephalopathies, Creutzfeldt-Jakob disease, Huntington's Chorea, ischemia, obsessive-compulsive disorder, manic depression, bipolar disorders, drug addiction, alcoholism and smoking addiction.

In other preferred embodiments, the methods and compositions of the present invention encompass the prevention and treatment of the dermatological disorders selected from the group consisting of acne, psoriasis, eczema, burns, poison ivy, poison oak and dermatitis.

In other preferred embodiments, the methods and compositions of the present invention encompass the prevention and treatment of the surgical disorders selected from the group consisting of pain and swelling following surgery, infection following surgery and inflammation following surgery.

In other preferred embodiments, the methods and compositions of the present invention encompass the prevention and treatment of the gastrointestinal disorders selected from the group consisting of inflammatory bowel syndrome, Crohn's disease, gastritis, irritable bowel syndrome, diarrhea, constipation, dysentery, ulcerative colitis, gastric esophageal reflux, ulcers, and heartburn.

In other preferred embodiments, the methods and compositions of the present invention encompass the prevention and treatment of the otic disorders selected from the group consisting of otic pain, inflammation, otorrhea, otalgia, fever, otic bleeding, Lermoyez's syndrome, Meniere's disease, vestibular neuronitis, benign paroxysmal positional vertigo, herpes zoster oticus, Ramsay Hunt's syndrome, viral neuronitis, ganglionitis, geniculate herpes, labyrinthitis, purulent labyrinthitis, viral endolymphatic labyrinthitis, perilymph fistulas, noise-induced hearing loss, presbycusis, drug-induced ototoxicity, acoustic neuromas, aerotitis media, infectious myringitis, bullous myringitis, otitis media, otitis media with effusion, acute otitis media, secretory otitis media, serous otitis media, acute mastoiditis, chronic otitis media, otitis extema, otosclerosis, squamous cell carcinoma, basal cell carcinoma, nonchromaffin paragangliomas, chemodectomas, globus jugulare tumors, globus tympanicum tumors, external otitis, perichondritis, aural eczematoid dermatitis, malignant external otitis, subperichondrial hematoma, ceruminomas, impacted cerumen, sebaceous cysts, osteomas, keloids, otalgia, tinnitus, vertigo, tympanic membrane infection, typanitis, otic furuncles, otorrhea, acute mastoiditis, petrositis, conductive and sensorineural hearing loss, epidural abscess, lateral sinus thrombosis, subdural empyema, otitic hydrocephalus, Dandy's syndrome, bullous myringitis, cerumen-impacted, diffuse external otitis, foreign bodies, keratosis obturans, otic neoplasm, otomycosis, trauma, acute barotitis media, acute eustachian tube obstruction, post-otic surgery, postsurgical otalgia, cholesteatoma, conductive and sensorineural hearing loss, epidural abscess, lateral sinus thrombosis, subdural empyema and otitic hydrocephalus.

In other preferred embodiments, the methods and compositions of the present invention encompass the prevention and treatment of the ophthalmic disorders selected from the group consisting of conjunctivitis, age-related macular degeneration diabetic retinopathy, detached retina, glaucoma, vitelliform macular dystrophy type 2, gyrate atrophy of the choroid and retina, conjunctivitis, corneal infection, fuchs' dystrophy, iridocorneal endothelial syndrome, keratoconus, lattice dystrophy, map-dot-fingerprint dystrophy, ocular herpes, pterygium, myopia, hyperopia, and cataracts.

For purposes of the present invention, the terms “diaryl-substituted pyrazolyl Cox-2 selective inhibitor”, “diaryl-substituted thiophenyl Cox-2 selective inhibitor”, “diaryl-substituted pyridyl Cox-2 selective inhibitor”, “diaryl-substituted isoxazolyl Cox-2 selective inhibitor”, “diaryl-substituted imidazolyl Cox-2 selective inhibitor” and “diaryl-substituted furanonyl Cox-2 selective inhibitor”, refer to any compound that selectively inhibits the Cox-2 enzyme relative to the cyclooxygenase-1 enzyme and comprises a structure having 2 aryl (or 1 heteroaryl and 1 aryl) rings, each of which is attached to (respectively) a pyrazolyl, thiophenyl, imidazolyl, isoxazolyl, oxazolyl, pyridyl, or furanonyl ring. As used herein, the terms “diaryl-substituted Cox-2 selective inhibitor” are to be understood to include all diaryl-substituted pyrazolyl, thiophenyl, imidazolyl, isoxazolyl, oxazolyl, pyridyl, or furanonyl Cox-2 selective inhibitors. While not intended to be limiting, several examples of known diaryl-substituted Cox-2 selective inhibitors include celecoxib, valdecoxib, parecoxib, deracoxib, rofecoxib, lumiracoxib, and etoricoxib.

In an embodiment of the present invention, the diaryl-substituted Cox-2 selective inhibitor can be a compound having the structure shown in formula I: 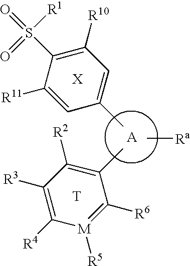
wherein the various rings and substituent groups will be described in more detail below.

The present invention takes advantage of certain positional halogen, haloalkyl, haloalkoxy, alkyl, nitrile, cyano or alkoxy substitutions on the active molecule itself to extend the duration of drug activity in a subject's blood plasma. The terms “extended duration”, and “extended effective half-life”, as they refer to a Cox 2 selective inhibitor, mean a Cox-2 selective inhibitor that has been modified according to the present invention and that has an effective half-life that is longer than that of the Cox-2 selective inhibitor prior to the modification.

The methods of the present invention differ from extended release or sustained release formulations that increase the duration of the effect of a drug by incorporating the drug into a matrix formulation for slow release into the blood plasma. The term “drug” is defined as a chemical capable of administration to an organism, which modifies or alters the organism's physiology. Instead, the present invention extends the duration of a diaryl-substituted Cox-2 selective inhibitor by adding certain substituent groups at various positions on the “T” ring (ring “T” in formulas I, II, and III) or “X” ring (ring “X” in formulas I, II, and III).

In a general sense, the rate of drug metabolism often determines the effective half-life for a drug. The terms “half-life,” “t_{1/2},” “effective half-life,” or “plasma half-life” mean the time after administration required for the plasma drug concentration or the amount of the drug in the body to decrease by 50%. Thus, drugs with a longer elimination half-life will retain activity at their primary site of action for an extended durational time requiring less frequent dosing.

As used herein, the terms “increase in plasma half-life” refer to the positive change in circulating half-life of a modified biologically active molecule relative to the circulating half-life of the biologically active molecule its non-modified form. Plasma half-life can be measured by taking blood samples at various time points after administration of the biologically active molecule, and determining the concentration of that molecule in each sample. Correlation of the plasma concentration with time allows calculation of the plasma half-life. The increase is preferably at least about two-fold, but a smaller increase may be useful, for example where it enables a satisfactory dosing regimen or avoids a toxic effect.

Therefore, in one embodiment, the present invention encompasses a novel method for increasing the plasma half-life of a Cox-2 selective inhibitor comprising adding one or more substituent groups onto either or both of the “T” ring and “X” ring of a Cox-2 selective inhibitor having the structure according to formulas I, II, or III described herein. It is preferred that the plasma half-life of the extended duration Cox-2 selective inhibitor in a rat model assay is at least 24 hours, more preferably at least 48 hours, and more preferably still, at least 72 hours.

In another embodiment, the present invention encompasses a novel therapeutic composition comprising an extended duration Cox-2 inhibitor having the structure according to formulas I, II, or III described herein. It is preferred that the plasma half-life of the composition in a rat model assay is at least 24 hours, more preferably at least 48 hours, and more preferably still, at least 72 hours.

In another embodiment, the present invention encompasses a novel method for increasing the plasma half-life of a Cox-2 selective inhibitor comprising adding one or more substituent groups onto either or both of the “T” ring and “X” ring of a Cox-2 selective inhibitor having the structure according to formulas I, II, or III described herein. It is preferred that the plasma half-life of the extended duration Cox-2 selective inhibitor in a rat model assay is from about 24 hours to about 1176 hours.

In a preferred embodiment, the plasma half-life of the extended duration Cox-2 selective inhibitor in a rat model assay is from about 36 hours to about 336 hours; more preferred is a plasma half-life of from about 48 hours to about 192 hours.

The present inventors have unexpectedly discovered that the introduction of a halogen, haloalkyl, haloalkoxy, alkyl, nitrile, cyano or alkoxy substitution can successfully be used to extend the duration of a daryl-substituted Cox-2 selective inhibitor's half-life in a subject. In particular, halogen, haloalkyl, haloalkoxy, alkyl, nitrile, cyano or alkoxy substitutions at certain positions on the “T” ring (ring “T” in formulas I, II, and III) or “X” ring (ring “X” in formulas I, II, and III), have been found to increase the effective half-life these compounds.

The present inventors have discovered that one of these compounds has an effective half-life persisting as long as 8 days in rats and 49 days in canines after only a single dosage administration. (e.g. 4-[5-(4-Chlorophenyl)-3-(trifluoromethyl)-1H-pyrazol-1-yl]benzenesulfonamide).

The present inventors have also unexpectedly discovered that the effective half-life for halogen, haloalkyl, haloalkoxy, alkyl, nitrile, cyano or alkoxy substituted diaryl-substituted Cox-2 selective inhibitors is influenced largely by the relative position of substitution and only to a lesser degree by the extent of substitution. From a positional perspective, substitution at the para position of the “T” ring (ring “T” in formulas I, II, and III) resulted in a marked reduction in metabolic clearance, thus giving extended duration, whereas substitution at the meta- or ortho position only resulted in a mild reduction in metabolic clearance. Substitution at a position ortho to the sulfonyl group of the “X” ring (ring “X” in formulas I, II, and III) also resulted in a effective reduction in metabolic clearance, thus also giving extended duration.

The longest increases in half-life arise from molecules that have halogen substituents in the “T” ring's (ring “T” in formulas I, II, and III) para position alone or in combination with halogen substituents either in the meta or ortho positions. The presence of a methyl group or methoxy group on the “T” ring in addition to a halogen at the para position also extends half-life, but not to the same extent as without the methyl or methoxy group. A methoxy or methyl at the para position and halogen substituents at the ortho or meta positions gives a smaller increase in effective half-life. Methoxy and/or methyl substitutions on the “T” ring give yet smaller increases in effective half-life.

Without wishing to be bound by a particular theory, increasing the number of halogen substitutions on the “T” ring (ring “T” in formulas I, II, and III) or “X” ring (ring “X” in formulas I, II, and III) is somewhat effective in increasing the effective half-life of the molecule. Overall, merely having the presence of at least one halogen, haloalkyl, haloalkoxy, alkyl, nitrile, cyano or alkoxy substituent covalently coupled to any carbon on the “T” ring or “X” ring was discovered to extend the plasma half-life of the entire molecule to some extent.

Although the following positional arrangements are listed by way of preferential order, this should not be construed as limiting the present invention, since all hydrogen, haloalkyl, haloalkoxy, alkyl, nitrile, cyano and alkoxy substitutions on the “T” ring and/or the “X” ring in formulas I, II, and III, are considered within the methods of the present invention. Likewise, even though the only halogens discussed below are chlorine, bromine, and fluorine, all other halogens are considered within the methods of the present invention. Still further embodiments include all diaryl substituted Cox-2 selective inhibitors in addition to the diaryl substituted pyrazolyl and isoxazolyl benzenesulfonamides classes of Cox-2 selective inhibitors.

In a one embodiment, the positional arrangement for the present invention has one or more of a halogen, haloalkyl, haloalkoxy, alkyl, nitrile, cyano or alkoxy substitutions at any position on the 4-pyrazolyl phenyl, 4-isoxazolyl phenyl, 4-furanonyl phenyl, 4-oxazolyl or 5-pyridyl phenyl rings (ring “T” in formulas I, II, and III).

In another embodiment, the positional arrangement for the present invention has one or more of a halogen, haloalkyl, haloalkoxy, alkyl, nitrile, cyano or alkoxy substitutions at any position on the “T” ring in formulas I, II, and III and at any position on the “X” ring in formulas I, II, and III.

In another embodiment, the positional arrangement for the present invention has one or more of a halogen or haloalkyl substitutions at any position on the “X” ring in formulas I, II, and III.

In yet another embodiment, the positional arrangement for the present invention has one or more of a halogen, haloalkyl, haloalkoxy, alkyl, nitrile, cyano or alkoxy substitutions at a position on the “X” ring that is ortho to the sulfonyl group on the same “X” ring in formulas I, II, and III.

In still another embodiment, the present invention has one or more of a halogen, haloalkyl, haloalkoxy, alkyl, nitrile, cyano or alkoxy substitutions at any of a para, meta or ortho position on the 4-pyrazolyl phenyl, 4-isoxazolyl phenyl, 4-furanonyl phenyl, 4-oxazolyl or 5-pyridyl phenyl rings (ring “T” in formulas I, II, and III).

In yet another embodiment, the present invention has one or more halogen substitutions at any of a para, meta or ortho position on the 4-pyrazolyl phenyl, 4-isoxazolyl phenyl, 4-furanonyl phenyl, or 5-pyridyl phenyl rings (ring “T” in formulas I, II, and III).

In yet another embodiment, the present invention has one or more halogen, haloalkyl, haloalkoxy, alkyl, nitrile, cyano or alkoxy substitutions at a para position (e.g. 4-chlorophenyl) on the 4-pyrazolyl phenyl, 4-isoxazolyl phenyl, 4-furanonyl phenyl, 4-oxazolyl or 5-pyridyl phenyl rings (ring “T” in formulas I, II, and III).

In another embodiment, the preferred positional arrangement for the present invention has a halogen at a para position and an alkyl or alkoxy group at a meta or ortho (or both) position on the ring “T” in formulas I, II, and III.

In another embodiment, the preferred positional arrangement for the present invention has a chlorine or a fluorine at the para and at the meta position (e.g. 3,4-chlorophenyl or 3,4-fluorophenyl).

In still another embodiment, the preferred positional arrangement has a chlorine or a fluorine at the para and the ortho position (e.g. 2,4-chlorophenyl or 2,4-fluorophenyl).

In yet another embodiment, the preferred positional arrangement has a chlorine, fluorine, or bromine at the para position and has an alkoxy group at the meta position. In another embodiment, the preferred positional arrangement is to have an alkoxy group at the para position in addition to at least one or more halogen substitutions at either the meta or ortho position.

In still another embodiment, the present invention has one or both of a chlorine or fluorine substitution at any or all of a para, meta, or ortho position on the ring “T” in formulas I, II, and III.

In still another embodiment, the present invention has a chlorine or fluorine substitution at a para position on the ring “T” in formulas I, II, and III. In yet another embodiment, the present invention has a chlorine substitution at a para position on the 4-pyrazolyl phenyl, 4-isoxazolyl phenyl, 4-oxazolyl, 4-furanone phenyl, or 5-pyridyl phenyl rings (e.g. 4-chlorophenyl on the “T” ring in formulas I, II, and III). In still another embodiment, the present invention has a halogen substitution at a position ortho to the sulfonyl group on ring “X” in formulas I, II, and III.

Other arrangements of halogen, alkyl and alkoxy substituents on the “T” ring and “X” rings in formulas I, II, and III, although not expressly recited, are still considered within the scope of the present invention.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). The term “IC₅₀” refers to the determination of enzyme binding affinity of a ligand using a competitive binding curve, the IC₅₀ (or EC₅₀—effective concentration 50%) is the concentration required for 50% inhibition. For purposes of the present invention, an extended duration Cox-2 selective inhibitor is any inhibitor for which the ratio of Cox-1 IC₅₀ to Cox-2 IC₅₀ is greater than 1, preferably greater than 2, more preferably greater than 5, yet more preferably greater than 10, still more preferably greater than 50, even more preferably greater than 100, still more preferably greater than 300, and even more preferably still greater than 500.

Preferred extended duration Cox-2 selective inhibitors of the present invention have a Cox-2 IC₅₀ of less than about 1 μM, more preferred of less than about 0.5 μM, and even more preferred of less than about 0.2 μM.

Preferred Cox-2 selective inhibitors have a Cox-1 IC₅₀ of greater than about 1 μM, and more preferably of greater than 20 μM.

In a preferred embodiment, the present invention encompasses a method of providing extended duration treatment of pain, inflammation and inflammation-related disorders in a subject in need of such extended duration treatment, the method comprising administering to the subject a compound having the formula I: 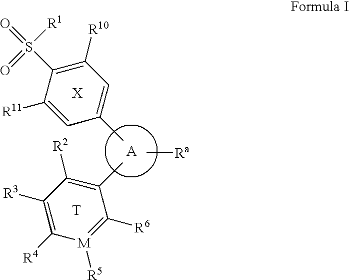
wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen; and
including the diastereomers, enantiomers, racemates, tautomers, salts, esters, amides and prodrugs thereof.

In another embodiment, the present invention comprises a method of providing extended duration treatment of pain, the method comprising administering to the subject in need of such therapy, a compound having the formula I, wherein:
R² and R⁶ are each independently selected from the group consisting of hydrogen and halo;
R³ and R⁵ are each independently selected from the group consisting of hydrogen, halo, alkyl and alkoxy; and
R⁴ is selected from the group consisting of halo, alkyl and alkoxy, except that when R⁴ is alkoxy, R³ is other than fluoro, when M is nitrogen, R⁴ is other than methyl, and when R⁴ is methyl, one of R², R³, R⁵ and R⁶ is other than hydrogen.

In yet another embodiment, the present invention comprises a method of providing extended duration treatment of pain, inflammation and inflammation-related disorders in a subject in need of such extended duration treatment, the method comprising administering to the subject a compound having the formula II: 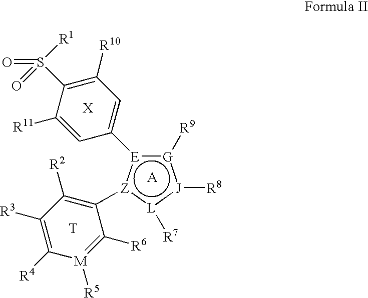
wherein:
E is selected from the group consisting of nitrogen, sulfur and carbon;
G is selected from the group consisting of nitrogen, sulfur, oxygen and carbon;
J is selected from the group consisting of nitrogen, sulfur, carbon and oxygen;
L is selected from the group consisting of nitrogen, oxygen and carbon;
Z is selected from the group consisting of nitrogen and carbon;
M is selected from the group consisting of nitrogen and carbon;
R¹ is selected from the group consisting of hydrogen, propanamide, amino and methyl;
R², R³, R⁵, R⁶, R¹⁰ and R¹¹ are optionally present and independently selected from the group consisting of hydrogen, cyano, nitrile, methyl, methoxy, fluorine, chlorine, bromine, iodine, astatine and haloalkyl;
R⁴ is optionally present and independently selected from the group consisting of hydrogen, cyano, nitrile, methyl, methoxy, fluorine, chlorine, bromine, iodine, astatine, haloalkyl and substituted or unsubstituted heterocycle, except that when R⁴ is methoxy, R³ is other than fluoro, when M is nitrogen, R⁴ is other than methyl, and when R⁴ is methyl, one of R², R³, R⁵ and R⁶ is other than hydrogen;
R⁷ is optionally present and is independently selected from the group consisting of hydrogen and oxygen;
R⁸ is optionally present and is independently selected from the group consisting of hydrogen, alkyl and haloalkyl;
R⁹ is optionally present and is independently selected from the group consisting of hydrogen, alkyl and haloalkyl; and
including diastereomers, enantiomers, racemates, tautomers, salts, esters, amides and prodrugs thereof.

In another embodiment, the present invention comprises a method of providing extended duration treatment of pain, inflammation and inflammation-related disorders in a subject in need of such extended duration treatment, the method comprising administering to the subject a compound having the formula III: 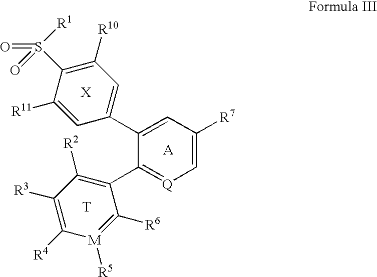
wherein:
M is selected from the group consisting of nitrogen and carbon;
Q is selected from the group consisting of nitrogen and carbon;
R¹ is selected from the group consisting of hydrogen, propanamide, and methyl;
R², R³, R⁵, R⁶, R¹⁰ and R¹¹ are optionally present and independently selected from the group consisting of hydrogen, cyano, nitrile, methyl, methoxy, fluorine, chlorine, bromine, iodine, astatine and haloalkyl;
R⁴ is optionally present and independently selected from the group consisting of hydrogen, cyano, nitrile, methyl, methoxy, fluorine, chlorine, bromine, iodine, astatine, haloalkyl and substituted or unsubstituted heterocycle, except that when R⁴ is methoxy, R³ is other than fluoro, when M is nitrogen, R⁴ is other than methyl, and when R⁴ is methyl, one of R², R³, R⁵ and R⁶ is other than hydrogen;
R⁷ is selected from the group consisting of hydrogen, chlorine, fluorine, bromine, iodine and astatine;
including diastereomers, enantiomers, racemates, tautomers, salts, esters, amides and prodrugs thereof.

In another embodiment, the present invention comprises a method of providing extended duration treatment of pain, inflammation and inflammation-related disorder the method comprising administering to the subject in need of such therapy, a compound having the formula I: 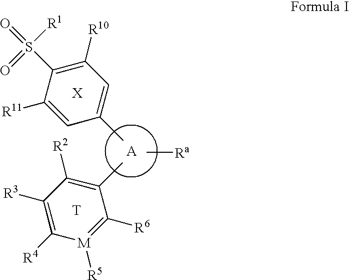
wherein:
R² and R⁶ are each independently selected from the group consisting of hydrogen and halo;
R³ and R⁵ are each independently selected from the group consisting of hydrogen, halo, alkyl and alkoxy;
R⁴ is selected from the group consisting of halo and alkoxy, except that when R⁴ is alkoxy, R³ is other than fluoro; and
R¹⁰ and R¹¹ are each independently selected from the group consisting of hydrogen and halo.

In another embodiment, the present invention comprises a method of providing extended duration treatment of pain, inflammation and inflammation-related disorder the method comprising administering to the subject in need of such therapy, a compound having the formula I, wherein:
R² and R⁶ are each independently selected from the group consisting of hydrogen, chlorine, bromine, iodine and fluorine;
R³ and R⁵ are each independently selected from the group consisting of hydrogen, chlorine, bromine, fluorine, methyl and methoxy;
R⁴ is selected from the group consisting of chlorine, bromine, iodine, fluorine and methoxy, except that when R⁴ is methoxy, R³ is other than fluoro; and
R¹⁰ and R¹¹ are each independently selected from the group consisting of hydrogen and halo.

In another embodiment, the present invention comprises a method of providing extended duration treatment of pain, inflammation and inflammation-related disorder the method comprising administering to the subject in need of such therapy, a compound having the formula I, wherein:
R² and R⁶ are each independently selected from the group consisting of hydrogen and halo;
R³ and R⁵ are each independently selected from the group consisting of hydrogen, halo, alkyl and alkoxy;
R⁴ is halo; and
R¹⁰ and R¹¹ are each independently selected from the group consisting of hydrogen and halo.

In another embodiment, the present invention comprises a method of providing extended duration treatment of pain, inflammation and inflammation related-disorder the method comprising administering to the subject in need of such therapy, a compound having the formula I, wherein:
R² and R⁶ are each independently selected from the group consisting of hydrogen, chlorine, bromine, iodine and fluorine;
R³ and R⁵ are each independently selected from the group consisting of hydrogen, chlorine, bromine, iodine, fluorine, methyl and methoxy;
R⁴ is selected from the group consisting of chlorine, iodine, fluorine and bromine; and
R¹⁰ and R¹¹ are each independently selected from the group consisting of hydrogen, chlorine, iodine, fluorine and bromine.

In another embodiment, the present invention comprises a method of providing extended duration treatment of pain, the method comprising administering to the subject in need of such therapy, a compound having the formula I, wherein:
R² and R⁶ are each independently selected from the group consisting of hydrogen and halo;
R³ and R⁵ are each independently selected from the group consisting of hydrogen, halo and alkyl;
R⁴ is halo; and
R¹⁰ and R¹¹ are each independently selected from the group consisting of hydrogen, chlorine, iodine, fluorine and bromine.

In another embodiment, the present invention comprises a method of providing extended duration treatment of pain, inflammation and inflammation related-disorder the method comprising administering to the subject in need of such therapy, a compound having the formula I, wherein:
R² and R⁶ are each independently selected from the group consisting of hydrogen, chlorine, fluorine, iodine and bromine;
R³ and R⁵ are each independently selected from the group consisting of hydrogen, chlorine, fluorine, iodine, bromine and methyl;
R⁴ is selected from the group consisting of chlorine, fluorine, iodine and bromine; and
R¹⁰ and R¹¹ are each independently selected from the group consisting of hydrogen, chlorine, iodine, fluorine and bromine.

In another embodiment, the present invention comprises a method of providing extended duration treatment of pain, inflammation and inflammation related-disorder the method comprising administering to the subject in need of such therapy, a compound having the formula I, wherein:
R² and R⁶ are each independently selected from the group consisting of hydrogen and halo;
R³ and R⁵ are each independently selected from the group consisting of hydrogen and halo;
R⁴ is halo; and
R¹⁰ and R¹¹ are each independently selected from the group consisting of hydrogen, chlorine, iodine, fluorine and bromine.

In another embodiment, the present invention comprises a method of providing extended duration treatment of pain, inflammation and inflammation related-disorder the method comprising administering to the subject in need of such therapy, a compound having the formula I, wherein:
R² and R⁶ are each independently selected from the group consisting of hydrogen, chlorine, fluorine, iodine and bromine;
R³ and R⁵ are each independently selected from the group consisting of hydrogen, chlorine, fluorine, iodine and bromine;
R⁴ is selected from the group consisting of chlorine, fluorine, iodine and bromine; and
R¹⁰ and R¹¹ are each independently selected from the group consisting of hydrogen, chlorine, iodine, fluorine and bromine.

In another embodiment, the present invention comprises a method of providing extended duration treatment of pain, inflammation and inflammation related-disorder the method comprising administering to the subject in need of such therapy, a compound having the formula I, wherein:
R² and R⁶ are each hydrogen;
R³ and R⁵ are each independently selected from the group consisting of hydrogen and halo;
R⁴ is halo; and
R¹⁰ and R¹¹ are each independently selected from the group consisting of hydrogen and halo.

In another embodiment, the present invention comprises a method of providing extended duration treatment of pain, inflammation and inflammation related-disorder the method comprising administering to the subject in need of such therapy, a compound having the formula I, wherein:
R² and R⁶ are each hydrogen;
R³ and R⁵ are each independently selected from the group consisting of hydrogen, chlorine, fluorine, iodine and bromine;
R⁴ is selected from the group consisting of chlorine, fluorine, iodine and bromine; and
R¹⁰ and R¹¹ are each independently selected from the group consisting of hydrogen, chlorine, iodine, fluorine and bromine.

In another embodiment, the present invention comprises a method of providing extended duration treatment of pain, inflammation and inflammation related-disorder the method comprising administering to the subject in need of such therapy, a compound having the formula I, wherein:
R⁴ is halo; and
R², R³, R⁵, R⁶, R¹⁰ and R¹¹ are each hydrogen.

In another embodiment, the present invention comprises a method of providing extended duration treatment of pain, inflammation and inflammation related-disorder the method comprising administering to the subject in need of such therapy, a compound having the formula I, wherein:
R⁴ is selected from the group consisting of chlorine, fluorine, bromine and iodine; and
R², R³, R⁵, R⁶, R¹⁰ and R¹¹ are each hydrogen.

In another embodiment, the present invention comprises a method of providing extended duration treatment of pain, inflammation and inflammation related-disorder the method comprising administering to the subject in need of such therapy, a compound having the formula I, wherein:
R⁴ is selected from the group consisting of chlorine and fluorine.

In another embodiment, the present invention comprises a method of providing extended duration treatment of pain, inflammation and inflammation related-disorder the method comprising administering to the subject in need of such therapy, a compound having the formula I, wherein:
R⁴ is chlorine, therefore making a preferred halogen substituent arrangement for purposes of the methods of the present invention a single chlorine substitution at the para position of the phenyl ring (e.g. 4-[5-(4-chlorophenyl)-3-(trifluoromethyl)-1H-pyrazol-1-yl]benzenesulfonamide).

In another embodiment, the present invention encompasses a therapeutic composition comprising a compound having the structure: 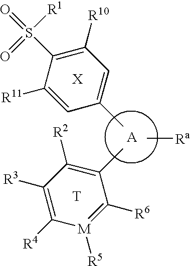
wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen; and
including the diastereomers, enantiomers, racemates, tautomers, salts, esters, amides and prodrugs thereof.

For the molecule (4-[5-(4-chlorophenyl)-3-(trifluoromethyl)-1H-pyrazol-1-yl]benzenesulfonamide), canine half-life (cassette-dosed) was surprisingly discovered to be around 1,189 hours or 49 days. Rat half-life was reported as 193 hours or 8 days. The molecule's selectivity was at least equivalent and in this particular circumstance, ten times greater than the compound, 4-[5-(4-methylphenyl)-3-(trifluoromethyl)-1H-pyrazol-1-yl]benzenesulfonamide. Thus, a single halogenation at the para position of the “T” ring (e.g. 4-chlorophenyl), was discovered to provide a compound having better selectivity for the Cox-2 enzyme and also to extend plasma half-life by at least 8 days and to as long as 49 days as compared to the compound, 4-[5-(4-methylphenyl)-3-(trifluoromethyl)-1H-pyrazol-1-yl]benzenesulfonamide.

For canines, test data for 4-[5-(4-methylphenyl)-3-(trifluoromethyl)-1H-pyrazol-1-yl]benzenesulfonamide indicates that effective half-life ranges from 1.72 hours (slow metabolizing subtype) to 5.72 hours (fast metabolizing subtype). See Paulson, S., et al. Drug Metab. Dispos. 28:308-314 (1999). In contrast, the 4-chlorophenyl substitution of the compound to provide 4-[5-(4-chlorophenyl)-3-(trifluoromethyl)-1H-pyrazol-1-yl]benzenesulfonamide, surprisingly extends the plasma half-life of the Cox-2 inhibitor in canines up to 1,189 hours. This constitutes a 207-fold increase in plasma half-life in canines for the Cox-2 selective inhibitor that was substituted according to the methods of the present invention as compared against the fast metabolizing subtype and a 691-fold increase as compared against the slow metabolizing subtype.

Thus, the compound, 4-[5-(4-chlorophenyl)-3-(trifluoromethyl)-1H-pyrazol-1-yl]benzenesulfonamide, demonstrates a maximum plasma concentration equivalent to an immediate release medicament (e.g. 4-[5-(4-methylphenyl)-3-(trifluoromethyl)-1H-pyrazol-1-yl]benzenesulfonamide) yet maintains a therapeutically effective blood concentration for a duration of at least 24 hours without the need for an increase in dosage strength or for additional dosing before expiration of the 24 hours. In fact, the compound, 4-[5-(4-chlorophenyl)-3-(trifluoromethyl)-1H-pyrazol-1-yl]benzenesulfonamide, maintains a therapeutically effective blood concentration for at least 8 days and as long as 49 days.

The surprising duration of these compounds is believed to result from a significant reduction in metabolic clearance as a result of the particular substitutions that are the subject of the invention. Therefore, the present invention provides methods for extending the plasma half-life of diaryl-substituted Cox-2 selective inhibitors by adding halogen, haloalkyl, haloalkoxy, alkyl, nitrile, cyano or alkoxy substituents or derivatives thereof at various positions on the phenyl rings thereof. As described above, more particularly preferred is to achieve extended duration by adding halogen, haloalkyl, haloalkoxy, alkyl, nitrile, cyano or alkoxy substituents or derivatives thereof on the 4-(pyrazolyl, isoxazolyl, pyridyl, oxazolyl or furanonyl) phenyl ring (e.g. the “T” ring of formulas I, II or III) and/or on the “X” ring on a diaryl-substituted pyrazolyl, isoxazolyl, pyridyl, oxazolyl or furanonyl Cox-2 selective inhibitor compound having a structure according to any of the formulas I, II, or III described herein.

Still more preferred is to add at least one halogen, haloalkyl, haloalkoxy, alkyl, nitrile, cyano or alkoxy substituent or derivative thereof at any or all of the para, meta or ortho positions on the “T” ring and/or ortho positions on the “X” ring of a diaryl-substituted pyrazolyl, isoxazolyl, pyridyl, oxazolyl or furanonyl Cox-2 selective inhibitor compound having a structure according to any of the formulas I, II, or III described herein.

In another embodiment, it is preferred to add at least one halogen substituent or derivatives thereof at a position ortho to the sulfonyl group on the “X” ring position of a diaryl-substituted pyrazolyl, isoxazolyl, pyridyl, oxazolyl or furanonyl Cox-2 selective inhibitor compound.

Even more preferred still is to add a chlorine or fluorine atom at the para position on the ‘T’ ring and/or on the “X” ring of a diaryl-substituted pyrazolyl, isoxazolyl, pyridyl, oxazolyl or furanonyl Cox-2 selective inhibitor compound having a structure according to any of the formulas I, II, or III described herein in order to achieve an extended duration of inflammation treatment.

Still further preferred is to add a chlorine atom at the para position on the “T” ring of the compound, 4-[5-phenyl-3-(trifluoromethyl)-1H-pyrazol-1-yl]benzenesulfonamide, which is designated as the “T” ring in formula I.

In one embodiment of the invention the extended duration Cox-2 selective inhibitor is of the pyrazolyl benzenesulfonamide structural class that is substituted with one or more halogen, haloalkyl, haloalkoxy, alkyl, nitrile, cyano or alkoxy groups along an aryl or heteroaryl ring thereof (e.g. the 4-pyrazolyl phenyl ring, hereinafter the “T” ring of formula I and/or the “X” ring), and even more preferably having the structure of any one of the compounds having a structure shown by the general formulas I or II, shown below, and possessing, by way of example and not to be construed as limiting, the structures disclosed in Table 1, including the diastereomers, enantiomers, racemates, tautomers, salts, esters, amides and prodrugs thereof.

In another embodiment of the invention, the extended duration Cox-2 selective inhibitor is of the pyridyl benzenesulfonamide structural class that is substituted with one or more halogen, haloalkyl, haloalkoxy, alkyl, nitrile, cyano or alkoxy groups along the non-sulfonamide ring thereof (e.g. the 4-pyrazolyl ring, hereinafter the “T” ring of formula I and/or the “X” ring), and even more preferably having the structure of any one of the compounds having a structure shown by the general formula III shown below.

In yet another embodiment of the invention the extended duration Cox-2 selective inhibitor is of the isoxazolyl benzenesulfonamide structural class that is substituted with one or more halogen, haloalkyl, haloalkoxy, alkyl, nitrile, cyano or alkoxy groups along the ‘T’ ring and/or “X” ring of formulas I, II and III, and even more preferably having the structure of any one of the compounds having a structure shown by the general formulas I or II shown below, and possessing, by way of example and not to be construed as limiting, the structures disclosed in Table 1, including the diastereomers, enantiomers, racemates, tautomers, salts, esters, amides and prodrugs thereof.

In one embodiment, substituted pyrazolyl benzenesulfonamides that can serve as extended-duration Cox-2 selective inhibitors for the methods of the present invention include substituted pyrazolyl benzenesulfonamide derivatives that are described in U.S. Pat. No. 5,466,823 to Talley et al. The synthesis of the substituted pyrazolyl benzenesulfonamides is also described in U.S. Pat. No. 5,466,823 to Talley et al.

In another embodiment, halogen, haloalkyl, haloalkoxy, alkyl, nitrile, cyano or alkoxy substituted isoxazolyl benzenesulfonamides that can serve as extended-duration Cox-2 selective for the methods of the present invention include the substituted isoxazolyl benzenesulfonamide derivatives that are described in U.S. Pat. No. 5,633,272 to Talley et al. and U.S. Pat. No. 5,859,257 to Talley et al. The synthesis of such extended duration Cox-2 selective inhibitors is also described in U.S. Pat. No. 5,633,272 to Talley et al. and U.S. Pat. No. 5,859,257 to Talley et al.

Examples of representative compositions that are suitable for the methods and compositions described herein include: 4-[5-(2,4-[difluorophenyl)-3-(trifluoromethyl)-1H-pyrazol-1-yl]benzenesulfonamide, 4-[5-(4-chlorophenyl)-3-(trifluoromethyl)-1H-pyrazol-1-yl]benzenesulfonamide, 4-[5-(2,4-[dichlorophenyl)-3-(trifluoromethyl)-1H-pyrazol-1-yl]benzenesulfonamide, 4-[5-(3,4-dichlorophenyl)-3-(trifluoromethyl)-1H-pyrazol-1-yl]benzenesulfonamide, 4-[5-(4-fluorophenyl)-3-(trifluoromethyl)-1H-pyrazol-1-yl]benzenesulfonamide, 4-[5-(3,4-difluorophenyl)-3-(trifluoromethyl)-1H-pyrazol-1-yl]benzenesulfonamide, 4-[5-(4-methoxyphenyl)-3-(trifluoromethyl)-1H-pyrazol-1-yl]benzenesulfonamide, 4-[5-(3-chloro-4-methoxyphenyl)-3(trifluoromethyl)-1H-pyrazol-1-yl]benzenesulfonamide, 4-[5-(3,5-dichloro-4-methoxyphenyl)-3-(trifluoromethyl)-1H-pyrazol-1-yl]benzenesulfonamide, 4-[5-(4-methoxy-3-methylphenyl)-3-(trifluoromethyl)-1H-pyrazol-1-yl]benzenesulfonamide, 4-[5-(4-methoxy-3,5-dimethylphenyl)-3-(trifluoromethyl)-1H-pyrazol-1-yl]benzenesulfonamide, 4-[5-(3-chloro-4-methylphenyl)-3-(trifluoromethyl)-1H-pyrazol-1-yl]benzenesulfonamide, 4-[5-(3,5-dichloro-4-methylphenyl)-3-(trifluoromethyl)-1H-pyrazol-1-yl]benzenesulfonamide, 4-[5-(4-chloro-3-methylphenyl)-3-(trifluoromethyl)-1H-pyrazol-1-yl]benzenesulfonamide, 4-[5-(4-chloro-3-methoxyphenyl)-3-(trifluoromethyl)-1H-pyrazol-1-yl]benzenesulfonamide, 4-[5-(4-bromo-3-methoxyphenyl)-3-(trifluoromethyl)-1H-pyrazol-1-yl]benzenesulfonamide and 4-[3-(4-chlorophenyl)-5-(trifluoromethyl)isoxazol-4-yl]benzenesulfonamide.

Any standard pharmacokinetic protocol can be used to determine the effective half-life (t_{1/2}) from a systemic drug concentration profile in mammals following administration of an extended duration Cox-2 selective inhibitor of the present invention, and thereby establish whether that formulation meets the pharmacokinetic criteria set out herein. The terms “systemic drug concentration” refers to a drug concentration in a subject's bodily fluids, such as plasma, serum or blood. The terms also include drug concentrations in tissues bathed by the systemic fluids, including the skin.

The increase in total systemic drug concentration is one way of defining an increase of drug bioavailability due to molecular substitutions to the original drug. Systemic drug concentrations are measured using standard in vitro or in vivo drug measurement techniques. One such measurement technique is where the increase in drug bioavailability attributable to the administration of an extended duration Cox-2 selective inhibitor as described herein, is determined by recording the total plasma drug concentrations over time after administration of the drug. See U.S. Pat. No. 6,028,054 to Benet, et al. As used herein, the term “drug bioavailability” is defined as the total amount of drug systemically available over time. Bioavailability depends upon a number of factors, including how a drug product is designed and manufactured, its physicochemical properties, and factors that relate to the physiology and pathology of the patient.

The plasma half-life determination for the formulations of the invention are done by administering the extended duration Cox-2 selective inhibitors to a subject and measuring the levels of the intact active ingredient in the blood plasma at different time intervals over an extended period of up to several weeks.

Plasma drug levels are, in one embodiment, measured using any high-performance liquid chromatographic procedure as known to one of ordinary skill in the art. See e.g. Matzke, G., et al., “Applied Pharmacokinetics: Principle of Therapeutic Drug Monitoring”; Applied Therapeutics, Inc., Vancouver, Wash., USA, 3^{rd} ed., 15-1 (1992). For determining blood plasma concentrations of celecoxib and such related compounds as those described herein extraction with a cation exchange/hydrophobic mixed mode solid-phase extraction column can be performed and followed by reversed phase HPLC according to the teachings of Paulson, S., et al., Drug Metab. Dispos. 28:308-314 (2000). Alternatively, other techniques involving extraction of the compounds with acetonitrile and analysis with high performance liquid chromatography and mass spectrometry may be utilized. See Paulson, S., et al., Xenobiotica 30:731-744 (2000). In addition, other techniques such as spectrophotometry, gas chromatography, liquid chromatography-mass spectrometry (LC-MS), and immunoassay techniques are utilized to measure a patient's plasma drug levels over time as known to one of skill in the art.

The increase in drug bioavailability is defined as an increase in the Area Under the Curve (AUC) in a plot of the plasma level of the drug versus time. AUC is the integrated measure of systemic drug concentrations over time in units of mass-time/volume. The AUC from time zero (the time of dosing) to time infinity (when no drug remains in the body) following the administration of a drug dose is a measure of the exposure of the patient to the drug. The Merck Manual, Sec. 22, Ch. 298, Drug Input and Disposition, 17^{th} Edition (1999). The AUC is directly proportional to the total amount of drug that reaches the systemic circulation.

The “effective amount” of a drug means the dose or amount of a biologically active molecule or conjugate thereof sufficient to exhibit a detectable therapeutic effect to be administered to a subject and the frequency of administration to the subject which is readily determined by one having ordinary skill in the art, by the use of known techniques and by observing results obtained under analogous circumstances.

Currently, many commercially available diaryl-substituted Cox-2 selective inhibitors such as pyrazolyl and isoxazolyl benzenesulfonamide type Cox-2 selective inhibitors are prescribed under a daily dosing regimen. The requirement for daily dosing arises from the fact that for most commercially available Cox-2 selective inhibitors of this class, the drug is metabolized to sub-therapeutic plasma levels within 24 hours of administration. Thus, a patient will often re-dose at least once every 24 hours in order to maintain therapeutic plasma levels of the drug.

However, by practicing the methods described herein, administration of such extended duration Cox-2 selective inhibitors would be required less frequently. For example, in some embodiments, the extended duration Cox-2 selective inhibitors are administered once every several days (e.g. dosing once every 2 days or longer). The ability to dose less frequently while maintaining a therapeutic effect has significant advantages for clinicians and their patients.

The dosing levels of the present extended duration compounds required for efficacy are similar to the dosing levels for other commercially available Cox-2 selective inhibitors. Therefore, the extended duration Cox-2 selective inhibitors described in the present invention would be expected to provide relief from inflammation for a longer period of time, e.g. up to several days or longer, following a single dose at a dosage level similar to other commercially available Cox-2 selective inhibitors.

The dose or effective amount to be administered to a subject and the frequency of administration to the subject can be readily determined by one of ordinary skill in the art by the use of known techniques and by observing results obtained under analogous circumstances. In determining the effective amount or dose, a number of factors are considered by the attending diagnostician, including, but not limited to, the potency and duration of action of the compounds used, the nature and severity of the illness to be treated, as well as the sex, age, weight, drug metabolism capabilities, general health and individual responsiveness of the subject to be treated, and other relevant circumstances. Thus, it is not possible to specify an exact effective amount in advance. However, the effective amount for a given situation can be determined by one of skill in the art.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages. Various delivery systems include capsules, tablets, food, and gelatin capsules, for example.

As used herein, the term “therapeutically effective” is intended to qualify the amount of each agent for use in therapy, which will achieve the goal of preventing, or improvement in the severity of, the disorder being treated, while avoiding adverse side effects typically associated with alternative therapies. An inflammatory symptom is considered ameliorated or improved if any benefit is achieved, no matter how slight. An amount of an extended duration Cox-2 selective inhibitor that causes a decrease in the frequency of incidence is “prophylactically effective”, where the term “prophylactic” refers to the prevention of disease, whereas the term “therapeutic” refers to the effective treatment of existing disease.

Although not intended to be limiting, several examples of the dosage requirements for the extended-duration Cox-2 selective inhibitors of the present invention are set forth herein, although the limits that are identified as being preferred may be exceeded if expedient.

The amount of the Cox-2 inhibitor that may be combined with the carrier materials to produce a single dosage form will vary depending upon the host treated and the particular mode of administration. For example, a formulation intended for the oral administration of humans may contain from 0.1 mg to 7 g of active agent compounded optionally with an appropriate and convenient amount of carrier material which may vary from about 5 to about 95 percent of the total composition.

The compositions of the present invention may contain the extended duration Cox-2 inhibitor active ingredient in the range of about 0.1 mg to 7 g, preferable in the range of about 0.5 mg to 2000 mg and most preferably between 1.0 mg and 500 mg. A dose of about 0.01 to 100 mg/kg body weight, preferably between about 0.1 and 50 mg/kg body weight, may be appropriate.

Dosage unit forms for the extended duration Cox-2 inhibitor will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

Those skilled in the art will appreciate that dosages may also be determined with guidance from Goodman & Gilman's The Pharmacological Basis of Therapeutics, Ninth Edition (1996), Appendix II, pp. 1707-1711.

The required frequency of dosing will depend upon the effective half-life of each halogen, haloalkyl, haloalkoxy, alkyl, nitrile, cyano or alkoxy substituted molecule. Thus, for purposes of the present invention, the extended duration compounds may be administered to a subject at a dosing frequency that may range from a daily basis for some lower durational compounds such as 4-[5-(4-chloro-3-methylphenyl)-3-(trifluoromethyl)-1H-pyrazol-1-yl]benzenesulfonamide or once every 49 days for longer extended durational compounds such as, 4-[5-(4-chlorophenyl)-3-(trifluoromethyl)-1H-pyrazol-1-yl]benzenesulfonamide. Thus, a compound may be administered on a regimen of several times per day, for example 1 to 4 times per day, preferably once or twice per day and even more preferably, once per two to five days, and even more preferably still, once per week. The dosage can be administered as a single dosage or in divided dosages.

In one embodiment, the effectiveness of a particular dosage of an extended duration Cox-2 inhibitor for preventing and treating a neoplasia disorder is determined by staging the disorder at multiple points during a subject's treatment. For example, once a histological diagnosis is made, staging (i.e., determination of the extent of disease) helps determine treatment decisions and prognosis. Clinical staging uses data from the patient's history, physical examination, and noninvasive studies. Pathologic staging requires tissue specimens.

Pathological staging is performed by obtaining a biopsy of the neoplasm or tumor. A biopsy is performed by obtaining a tissue specimen of the tumor and examining the cells microscopically. A bone marrow biopsy is especially useful in determining metastases from malignant lymphoma and small cell lung cancer. Marrow biopsy will be positive in 50 to 70% of patients with malignant lymphoma (low and intermediate grade) and in 15 to 18% of patients with small cell lung cancer at diagnosis. See The Merck Manual of Diagnosis & Therapy, Beers & Brakow, 17^{th} edition, Published by Merck Research Labs, Sec. 11, Chapter 84, Hematology and Oncology, Overview of Cancer (1999).

Determination of serum chemistries and enzyme levels may also help staging. Elevation of liver enzymes (alkaline phosphatase, LDH, and ALT) suggests the presence of liver metastases. Elevated alkaline phosphatase and serum calcium may be the first evidence of bone metastases. Elevated acid phosphatase (tartrate inhibited) suggests extracapsular extension of prostate cancer. Fasting hypoglycemia may indicate an insulinoma, hepatocellular carcinoma, or retroperitoneal sarcoma. Elevated BUN or creatinine levels may indicate an obstructive uropathy secondary to a pelvic mass, intrarenal obstruction from tubular precipitation of myeloma protein, or uric acid nephropathy from lymphoma or other cancers. Elevated uric acid levels often occur in myeloproliferative and lymphoproliferative disorders. α-Fetoprotein may be elevated in hepatocellular carcinoma and testicular carcinomas, carcinoembryonic antigen-S in colon cancer, human chorionic gonadotropin in choriocarcinoma and testicular carcinoma, serum immunoglobulins in multiple myeloma, and DNA probes (bcr probe to identify the chromosome 22 change) in CML.

Tumors may synthesize proteins that produce no clinical symptoms, e.g., human chorionic gonadotropin, α-fetoprotein, carcinoembryonic antigen, CA 125, and CA 153. These protein products are used as tumor markers in the serial evaluation of patients for determining disease recurrence or response to therapy. Thus, monitoring a subject for these tumor markers is indicative of the progress of a neoplasia disorder. Such monitoring is also indicative of how well the methods and compositions of the present invention are treating or preventing a neoplasia disorder. Likewise, tumor marker monitoring is effective to determine the appropriate dosages of the compositions of the present invention for treating neoplasia.

Other techniques include mediastinoscopy, which is especially valuable in the staging of non-small cell lung cancer. If mediastinoscopy shows mediastinal lymph node involvement, then the subject would not usually benefit from a thoracotomy and lung resection. Imaging studies, especially CT and MRI, can detect metastases to brain, lung, spinal cord, or abdominal viscera, including the adrenal glands, retroperitoneal lymph nodes, liver, and spleen. MRI (with gadolinium) is the procedure of choice for recognition and evaluation of brain tumors.

Ultrasonography is used to study orbital, thyroid, cardiac, pericardial, hepatic, pancreatic, renal, and retroperitoneal areas. It may guide percutaneous biopsies and differentiate renal cell carcinoma from a benign renal cyst. Lymphangiography reveals enlarged pelvic and low lumbar lymph nodes and is useful in the clinical staging of patients with Hodgkin's disease, but it has generally been replaced by CT.

Liver-spleen scans can identify liver metastases and splenomegaly. Bone scans are sensitive in identifying metastases before they are evident on x-ray. Because a positive scan requires new bony formation (i.e., osteoblastic activity), this technique is useless in neoplasms that are purely lytic (e.g., multiple myeloma); routine bone x-rays are the study of choice in such diseases. Gallium scans can help in staging lymphoid neoplasms. Radiolabeled monoclonal antibodies (e.g., to carcinoembryonic antigen, small cell lung cancer cells) provide important staging data in various neoplasms (e.g., colon cancer, small cell lung cancer). See The Merck Manual of Diagnosis & Therapy, Beers & Brakow, 17^{th} edition, Published by Merck Research Labs, Sec. 11, Chapter 84, Hematology and Oncology, Overview of Cancer (1999).

In another preferred embodiment, the effectiveness of a particular dosage of an extended duration Cox-2 inhibitor for treating or preventing a cardiovascular disorder is determined and adjusted based on the efficacy demonstrated in reducing or preventing the symptoms of any cardiovascular disorder. In addition, one of ordinary skill in the art will know how to measure and quantify the presence or absence of cardiovascular disorder symptoms. For example, the degree and severity of hypertension can be determined by measuring the blood (arterial) pressure in the brachial artery of the arm. Blood pressure is equal to the total cardiac output multiplied by the total peripheral resistance. The systolic pressure occurs as the heart's ventricles contract and force blood into the aorta. The diastolic pressure occurs after the aortic valve closes and the pressure falls to a minimum level before the next ventricular contraction. This minimum pressure is the diastolic pressure. Hypertension occurs when the systolic and diastolic pressures depart from a normal value.

For most adults, normal blood pressures are typically less than 140 mm Hg for the systolic pressure and less than 85 mm Hg for the diastolic pressure. Pressures that exceed either of the two measurements are considered abnormal. Stage I hypertension (mild) is where the systolic pressure is between 140-159 mm Hg and the diastolic pressure is between 90-99 mm Hg. This range progresses until stage 1V hypertension (very severe) is reached where the systolic pressure is greater than 210 mm Hg or the diastolic pressure is greater than 120 mm Hg.

One example of an instrument used to measure arterial blood pressure is called a sphygmomanometer. For this procedure, a rubber inflatable cuff is place over the brachial artery and the pressure in the cuff is raised until the cuff pressure exceeds that of the blood in the artery. At this point, there is no blood flow and thus no pressure. The pressure in the cuff is then slowly released and the radial pulse then reappears. The pressure at which point the pulse reappears corresponds to the systolic pressure. Alternatively, a stethoscope can be used to listen for the (Korotkov) sounds of the pressure returning within the brachial artery (systolic) and the loss of sounds of when the pressures returns in full (diastolic).

Other procedures may be employed to determine the presence or absence of a cardiovascular disorder besides measuring the arterial blood pressure. For example, several invasive and noninvasive methods may be employed to diagnose and monitor such conditions as heart failure, congestive heart failure, myocardial infarction, myocardial fibrosis, and arteriosclerosis. See The Merck Manual, 17^{th} edition, Sec. 16, Chapter 198, Diagnostic Cardiovascular Procedures.

Examples of noninvasive techniques include plain radiography of the chest to determine heart size and shape, radionulceotide imaging, myocardial perfusion imaging to determine arterial stenosis, and magnetic resonance imaging. Id. Likewise, examples of invasive techniques include venous and arterial catheterization to accurately determine blood pressures; cardiac catheterization to determine heart anatomical information and blood flow data, angiocardiography, and angioplasty to revascularize coronary arteries. Id. Anyone of ordinary skill in the art can perform and interpret these procedures to determine the effectiveness of the methods and compositions of the present invention.

Still other techniques may be employed to determine the presence or absence of cardiovascular disorders after treatment with the methods and compositions of the present invention. The severity of heart failure is classified by a system established by the New York Heart Association (NYHA). The system is divided into four classes that are based on the degree of breathlessness to indicate a severity score. For example, Class I: the patient is breathless with more than ordinary activity, Class II: breathless with ordinary activity, Class III: breathless with minimal activity, and Class IV: breathless symptoms at rest. See Chavey, W., et al., Am. Fam. Physician 1;64(5):769-74 (2001). Thus, for example, a change from class IV to class III is indicative of an improvement in the symptoms of heart failure.

In addition, one of ordinary skill can determine the efficacy of the extended duration compositions of the present invention by monitoring the physiological levels of several biological markers. For example, changes in the levels of such markers as neurohormones including natriuretic peptides, urinary aldosterone, plasma renin, and serum potassium are indicative of how well the extended duration compositions provided in the present invention are treating or preventing a cardiovascular disorder. Natriuretic peptides are a group peptides that have diverse actions in cardiovascular, renal and endocrine homeostasis. Elevated natriuretic peptide levels in the blood are generally observed in subjects under conditions of blood volume expansion and after vascular injury such as acute myocardial infarction and remain elevated for an extended time after the infarction. See U.S. Pat. No. 6,410,524 to Perez, et al. and see Uusimaa, et al., Int. J. Cardiol. 69:5-14 (1999).

Thus, blood concentrations of natriuretic peptides are measured before and after administration of the extended duration therapy of the present invention in order to correlate the efficacy of a particular dosage with a reduction in the symptoms of a cardiovascular disorder. Accordingly, dosing of therapeutic compositions for cardiovascular disorders may be determined and adjusted based on measurement of blood concentrations of natriuretic peptides.

In yet another preferred embodiment, the effectiveness of a particular dosage of an extended duration Cox-2 inhibitor for treating or preventing a respiratory disorder is determined and adjusted based on the efficacy demonstrated in reducing or preventing the symptoms of any respiratory disorder. One of ordinary skill in the art will know how to measure and quantify the symptoms of inflammation. For example, the degree and severity of asthma and COPD can be determined by measuring lung expiratory flow volume and expiratory flow rates. Such a measurement is accomplished with, for example, a spirometer, flow volume loop, or pneumotach, before and after each of the treatments. Use of spirometry is a standard test for determining the efficacy of Cox-2 inhibitors and corticosteroids after administration to a subject suffering from a pulmonary inflammatory disorder. A device called a spirometer is used to measure how much air the lungs can hold and how well the respiratory system is able to move air into and out of the lungs.

Spirometry is a medical test that measures the physical volume of air an individual forcibly inhales or exhales into a device. The objective of spirometry is to assess ventilatory function. An estimate of flow rate, or the rate at which the volume is changing as a function of time can also be calculated with spirometery. See College of Physicians and Surgeons of Alberta, “Guidelines For Spirometry & Flow Volume Measurements” (1998). Thus, with the methods of the present invention, spirometric comparisons of pulmonary airflow before and after treatment will elucidate similarities and differences that enable one of skill to determine the effectiveness of the treatment methods.

Common parameters that spirometry measures are Forced Vital Capacity (FVC)—the maximum volume of air, measured in liters that can be forcibly and rapidly exhaled. Another parameter is Forced Expiratory Volume (FEV1)—the volume of air expelled in the first second of a forced expiration. Normal parameters for a subject not suffering from an inflammatory disorder such as asthma or COPD is: Tidal volume—5 to 7 milliliters per kilogram of body weight; Expiratory reserve volume—25% of vital capacity; Inspiratory capacity—75% of vital capacity forced expiratory volume—75% of vital capacity after 1 second, 94% after 2 seconds, and 97% after 3 seconds. Healthatozcom, wellness, test & procedures, spirometry <http://www.healthatoz.com/atoz/TestProcedures/TPspirometry.html>.

Spirometry results are expressed as a percentage, and are considered abnormal if less than 80% of the normal predicted value. An abnormal result usually indicates the presence of some degree of obstructive lung disease such as COPD and chronic bronchitis, or restrictive lung disease such as pulmonary fibrosis or asthma.

In another preferred embodiment, the effectiveness of a particular dosage of an extended duration Cox-2 inhibitor for treating or preventing an otic disorder is determined and adjusted based on the efficacy demonstrated in reducing or preventing the symptoms of any otic disorder.

For example, one method to detect whether a subject is suffering from an otic disorder, such as an ear infection, is to look in the ear with an otoscope. An otoscope is a lighted instrument that allows the physician to examine the outer ear and the eardrum. Redness or swelling of the eardrum is typical of an inflammatory response due to an infection.

Another method to diagnose and monitor an otic disorder is to check for the presence of middle ear fluid. The use of a special type of otoscope, called a pneumatic otoscope, allows the physician to blow a puff of air onto the eardrum to test eardrum movement. An eardrum with fluid build-up behind it does not move as well as an eardrum with air behind it.

Yet another method is to employ a test of middle ear function called tympanometry. This test requires insertion of a small soft plug into the opening of the subject's ear canal. The plug contains a speaker, microphone and a device that is able to change the air pressure in the ear canal, allowing for several measures of middle ear function. The subject feels air pressure changes in the ear or hears a few brief tones. While this test provides information on the condition of the middle ear, it does not determine how well the subject hears. An audiologist, a person who is specially trained to measure hearing, can also perform a hearing test. Both tests are indicative of the progress of treating an otic disorder.

The extended duration diaryl-substituted Cox-2 selective inhibitors of the present invention can be supplied as pure compounds, or in the form of a pharmaceutically acceptable salt. The extended duration compounds can also be supplied in the form of a prodrug, an isomer, a racemic mixture, or in any other chemical form or combination that, under physiological conditions, still provides for the extended duration of efficacy.

The compounds of the present invention can be supplied in the form of a pharmaceutically acceptable salt. The terms “pharmaceutically acceptable salt” refer to salts prepared from pharmaceutically acceptable inorganic and organic acids and bases.

Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary salts include aluminum, calcium, lithium, magnesium, potassium, sodium and zinc. Particularly preferred are the ammonium, calcium, magnesium, potassium, and sodium salts.

Salts derived from pharmaceutically acceptable organic non-toxic bases include salts of primary, secondary, and tertiary amines, including in part, trimethylamine, diethylamine, N,N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Illustrative pharmaceutically acceptable acid addition salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearid, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Exemplary pharmaceutically acceptable salts include the salts of hydrochloric acid and trifluoroacetic acid.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference only with regards to the disclosures of pharmaceutically acceptable salts.

The extended-duration compositions of the present invention may be administered to a patient in need of such therapy alone or in combination with a pharmaceutically acceptable carrier. A “pharmaceutically acceptable carrier” or “pharmaceutically acceptable excipient,” used interchangeably herein, includes, but is not limited to, physiological saline, Ringer's solution, phosphate solution or buffer, buffered saline and other carriers known in the art. Pharmaceutically acceptable carriers may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective. In one embodiment, the extended duration Cox-2 selective inhibitors are administered to a subject together in one pharmaceutical carrier. In another embodiment, they are administered separately.

The pharmaceutical compositions may be administered enterally and parenterally. In one embodiment, the compositions of the present invention are administered orally (intra-gastric). In another embodiment, the compositions of the present invention are injected intramuscularly or intravenously.

Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs. Topical dosage forms for administration of this invention include ointments, powders, sprays, inhalants, creams, jellies, collyriums, solutions or suspensions.

Parenteral administration includes subcutaneous, intramuscular, intradermal, intramammary, intravenous, and other administrative methods known in the art. Enteral administration includes solution, tablets, sustained release capsules, enteric-coated capsules, and syrups. When administered, the pharmaceutical composition may be at or near body temperature.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, sucrose, cellulose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract. For example, a material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the extended duration Cox-2 selective inhibitors may be formulated with sweetening agents, for example glycerol, sorbitol, or sucrose. Such formulations may also contain a demulcent, a preservative and flavoring and coloring agents.

The subject methods and compositions of extended duration Cox-2 selective inhibitors can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Also, administration can be by inhalation, in the form of aerosols or solutions for nebulizers, or rectally, in the form of suppositories prepared by mixing the drug with a suitable non-irritating excipient which is solid at ordinary temperature, but liquid at the rectal temperature and will therefore, melt in the rectum to release the drug. Such materials are cocoa butter and polyethylene glycols.

Pharmaceutically acceptable excipients and carriers encompass all the foregoing and the like. The above considerations concerning effective formulations and administration procedures are well known in the art and are described in standard textbooks. See e.g. Gennaro, A. R., Remington: The Science and Practice of Pharmacy, 20^{th} Edition, (Lippincott, Williams and Wilkins), 2000.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims that follow the examples.

### EXAMPLE 1

This example illustrates the synthesis and testing of extended duration Cox-2 selective inhibitors by methods according to the present invention.

Several halogen, haloalkyl, haloalkoxy, alkyl, nitrile, cyano or alkoxy substituted Cox-2 selective inhibitor drugs were synthesized by the methods described in, for example, U.S. Pat. No. 5,466,823 to Talley, et. al. Each of the compounds were evaluated for their specificity (IC₅₀ in μM) to the Cox-1 and Cox-2 enzymes, selectivity for the Cox-2 enzyme (Cox-1 IC₅₀/Cox-2 IC₅₀), and half-life (t_{1/2}) in rats and canines. The results of these assays are shown in table 1 below.

The specificity of a given extended duration Cox-2 inhibitor is measured by determining the IC₅₀ or EC₅₀ for each compound described above. The term “IC₅₀” refers to the determination of enzyme binding affinity of a ligand using a competitive binding curve, the IC₅₀ (or EC₅₀-effective concentration 50%) is the concentration required for 50% inhibition reported in μM. For purposes of the present invention, the enzymes are Cox-1 and Cox-2 and the ligand is one of the extended duration compounds described herein. The IC₅₀ and, thus, the Cox-1 and Cox-2 enzyme binding affinity for the extended duration compounds of the present invention is determined according to the teachings of Mardini, I., et al., Molecular Interventions 1:1 (2001.

For the purposes of this invention, the selectivity of the extended duration Cox-2 inhibitors described above is measured as a ratio of the in vitro or in vivo IC₅₀ value for inhibition of Cox-1, divided by the IC₅₀ value for inhibition of Cox-2 (Cox-1 IC₅₀/Cox-2 IC₅₀).

The extended duration compounds of the present invention were assayed in rats and canines. The 4-[5-(4-methylphenyl)-3-(trifluoromethyl)-1H-pyrazol-1-yl]benzenesulfonamide data is included for comparison purposes. As used in the table 1, “ND” refers to not determined. All rat pharmacokinetics data is non-cassette dosed. Non-cassette dosed pharmacokinetics means that each compound was dosed individually (i.e. not part of a cassette of compounds). Cassette-dosing is a technique in which 2 or more compounds are simultaneously administered to an animal and the pharmacokinetics of each compound is monitored individually even though it is in the same animal/blood sample. Where multiple IC₅₀'s are listed for a particular compound, each IC₅₀ is a repeated experimental result.

TABLE 1
Selected Properties of Extended Duration Cox-2 Selective Inhibitors

| | | | | hCOX | | | |
|---|---|---|---|---|---|---|---|
| | | hCOX-1 | hCOX-2 | selectivity | | Canine | |
| | | IC₅₀ | IC₅₀ | hCOX-1/ | Rat t_{1/2} | Cassette- | Canine t_{1/2} |
| Compound | Structure | (μM) | (μM) | hCOX-2 | (hr) | dosed t_{1/2} (hr) | (hr) |
| 4-[5-(4-methylphenyl)-3- (trifluoromethyl)-1H- pyrazol-1- yl]benzenesulfonamide | 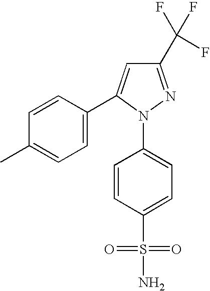 | >1.5 | <.004 | ˜375 | 3.73 in Male 14.0 in Female | ND | 1.72-5.18 |
| 4-[5-phenyl-3-- (trifluoromethyl)-1H- pyrazol-1- yl]benzenesulfonamide | 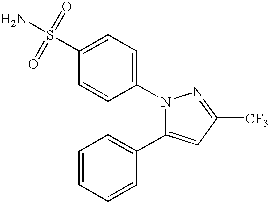 | 112 55.1 50.6 | 0.103 0.032 <0.137 | 1703 | 7.2 | 24 | 6-9 |
| 4-[5-(4-chlorophenyl)-3- (trifluoromethyl)-1H- pyrazol-1- yl]benzenesulfonamide | 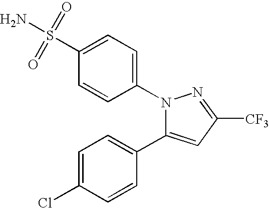 | 15.0 21.0 | 0.004 0.006 | 3750 | 193 | ˜1189 | ND |
| 4-[5-(3,4- dichlorophenyl)-3- (trifluoromethyl)-1H- pyrazol-1- yl]benzenesulfonamide | 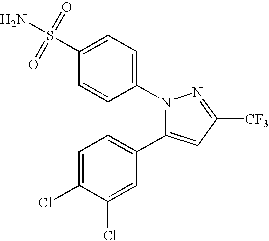 | 13.1 13.5 17.4 | 0.223 <0.0152 0.015 | 1160 | 157 | ˜830 | 762 |
| 4-[5-(2,4- [dichlorophenyl)-3- (trifluoromethyl)-1H- pyrazol-1- yl]benzenesulfonamide | 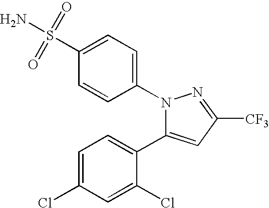 | 34.6 31.2 | 0.222 0.056 | 554 | 97 | ˜771 | 466 |
| 4-[5-(4-fluorophenyl)-3- (trifluoromethyl)-1H- pyrazol-1- yl]benzenesulfonamide | 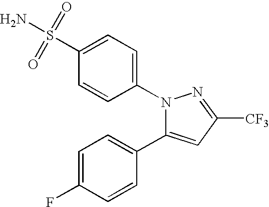 | 76.1 25.5 | 0.0515 0.041 | 1478 | 32 | 572 | 289 |
| 4-[5-(3,4- difluorophenyl)-3- (trifluoromethyl)-1H- pyrazol-1- yl]benzenesulfonamide | 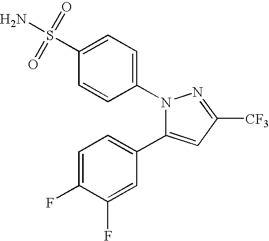 | >100 >500 73.2 | 0.216 0.396 <0.137 | ND | 59 | ˜881 | 500 |
| 4-[5-(2,4- [difluorophenyl)-3- (trifluoromethyl)-1H- pyrazol-1- yl]benzenesulfonamode | 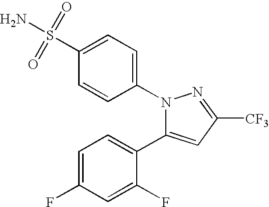 | 38.4 14 22.4 | 0.149 0.036 <0.137 | 393 | 25 | ˜716 | 233 |
| 4-[5-(4-chloro-3- methylphenyl)-3- (trifluoromethyl)-1H- pyrazol-1- yl]benzenesulfonamide | 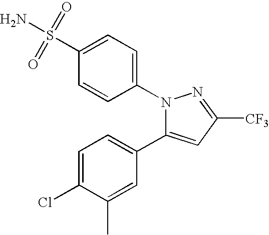 | 4.92 | <0.137 | ND | ND | ˜44 | 12 |
| 4-[5-(4-chloro-3- methoxyphenyl)-3- (trifluoromethyl)-1H- pyrazol-1- yl]benzenesulfonamide | 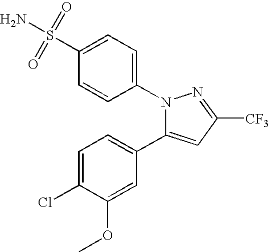 | 26.8 | 0.0072 | 1111 | ND | ˜104 | 48 |
| 4-[5-(4-bromo-3- methoxyphenyl)-3- (trifluoromethyl)-1H- pyrazol-1- yl]benzenesulfonamide | 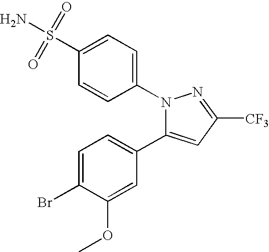 | 30.3 35.3 | 0.0453 <0.137 | 669 | ND | ND | 71 |
| 4-[5-(4-methoxyphenyl)- 3-(trifluoromethyl)-1H- pyrazol-1- yl]benzensulfonamide | 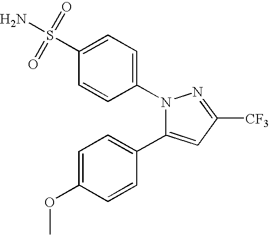 | 0.917 18.6 | <0.0051 >1.2 | ND | ND | ˜21 | ND |
| 4-[5-(3-chloro-4- methoxyphenyl)-3- (trifluoromethyl)-1H- pyrazol-1- yl]benzenesulfonamide | 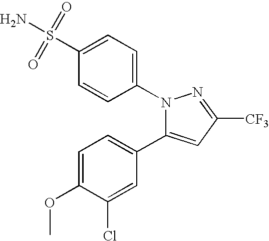 | 23.4 19.6 | 0.0165 0.0171 | 1418 | ND | ˜22 | ND |
| 4-[5-(3,5-dichloro-4- methoxyphenyl)-3- (trifluoromethyl)-1H- pyrazol-1- yl]benzensulfonamide | 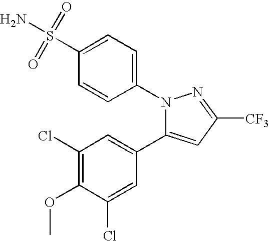 | 300 >500 | 0.0547 0.0390 | 5484 | ND | ˜17 | ND |
| 4-[5-(4-methoxy-3- methylphenyl)-3- (trifluoromethyl)-1H- pyrazol-1- yl]benzenesulfonamide | 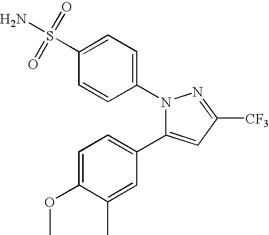 | 22.0 7.74 | 0.006 <0.004 | 3667 | ND | ˜36 | ND |
| 4-[5-(4-methoxy-3,5- dimethylphenyl)-3- (trifluoromethyl)-1H- pyrazol-1- yl]benzenesulfonamide | 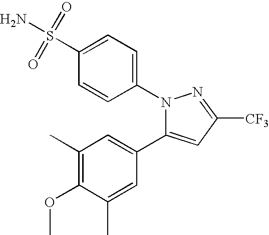 | >100 | 0.0800 | ND | ND | ˜18 | ND |
| 4-[5-(3-chloro-4- methylphenyl)-3- (trifluoromethyl)-1H- pyrazol-1- yl]benzenesulfonamide | 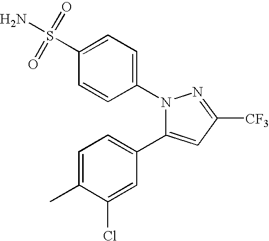 | 57.0 12.5 14.3 | 0.0249 <0.137 0.007 | 2289 | 5.1 | 17 | ND |
| 4-[5-(4-chloro-3- methoxyphenyl)-3- (trifluoromethyl)-1H- pyrazol-1- yl]benzenesulfonamide | 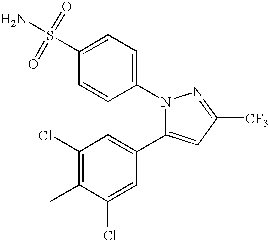 | 270 | 0.193 | 1399 | ND | ˜38 | ND |

### EXAMPLE 2

This example illustrates the pharmacokinetics testing in rats for the diaryl-substituted extended duration Cox-2 selective inhibitors of the present invention.

The extended duration Cox-2 selective inhibitor compounds tested in this example were synthesized according to the methods described in, for example, U.S. Pat. No. 5,466,823 to Talley, et. al. In this experiment halogen, the halogen, haloalkyl, haloalkoxy, alkyl, nitrile, cyano or alkoxy substituted pyrazolyl benzenesulfonamides of the present invention were dissolved in an aqueous polyethylene glycol solution and orally administered to rats by gastric gavage at a dosage of 2 mg/kg. At each sample collection time, blood samples were collected from three rats by cardiac puncture and then processed into plasma. Blood samples were collected up to 336 hours after dosing. Plasma drug concentrations were then determined by liquid chromatography-mass spectrometry methods. The mean plasma concentrations from each of the three samples collected at each time point were then plotted as a function of time. This process was repeated for each compound tested.

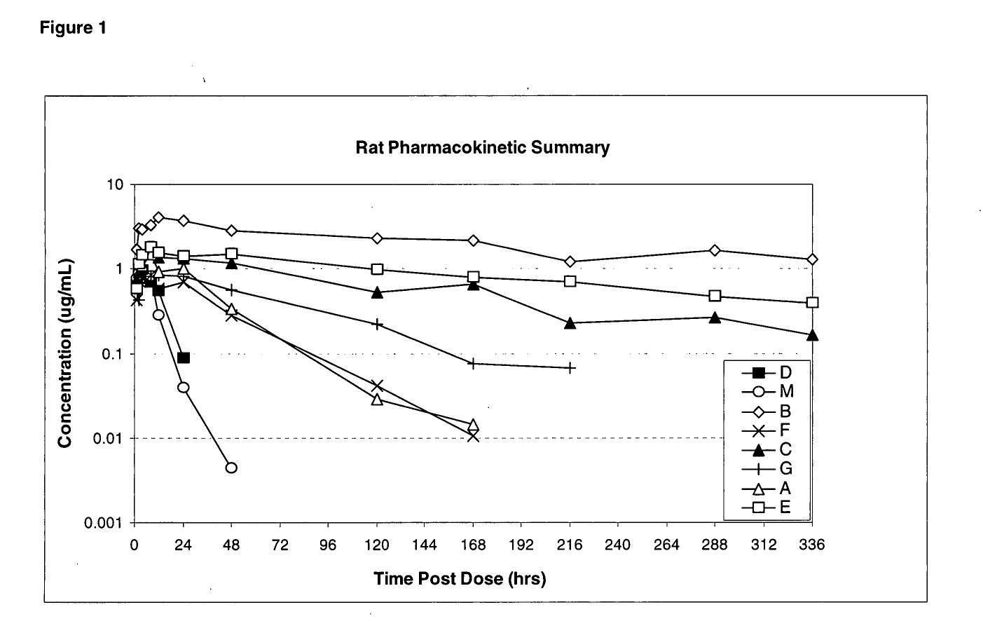

The graph in FIG. 1 corresponds to this experiment and shows mean plasma concentrations plotted as a function of time of the Cox-2 selective inhibitor (D-4-[5-phenyl-3-(trifluoromethyl)-1H-pyrazol-1-yl]benzenesulfonamide) in conjunction with the halogen, haloalkyl, haloalkoxy, alkyl, nitrile, cyano and alkoxy substituted Cox-2 selective inhibitors of the present invention. From the results, it is noted that several extended duration Cox-2 selective inhibitors maintain high plasma concentrations for days following a single oral dose. The plasma half-lives (t₁₂) determined in this example are provided in Table 1.

### EXAMPLE 3

This example illustrates an experiment performed to assay the effectiveness of halogen, haloalkyl, haloalkoxy, alkyl, nitrile, cyano or alkoxy substituted Cox-2 selective inhibitors of the present invention in reducing adjuvant-induced arthritis in rat paws.

This experiment was performed following a modified method of Connor, J. et al. (European J. Pharmacology, 1995, 273:15-24). Briefly, arthritis was induced in pathogen-free, male Lewis rats by an intadermal injection of 20 mg/ml Mycobacterium butyricum suspended in squalene in three sites at the base of the tail. The injection of mycobacteria produces a systemic autoimmune response and subsequent chronic inflammation of the joints with many similarities to rheumatoid arthritis. The induced arthritis was allowed to develop for 14 days at which time animals were given a single oral dose of a Cox-2 selective inhibitor at 3 mg/mL. Effectiveness was assessed by the decrease in rat paw edema (paw volume) for 28 days following the Cox-2 inhibitor dose. Paw volume was determined using a water displacement plethysmometer. To gage the drug response, the paw volumes of rats treated with Cox-2 selective inhibitors were compared to normal healthy rats without induced arthritis and without drug (normal-control group) and rats with induced arthritis but without drug (adjuvant-control group).

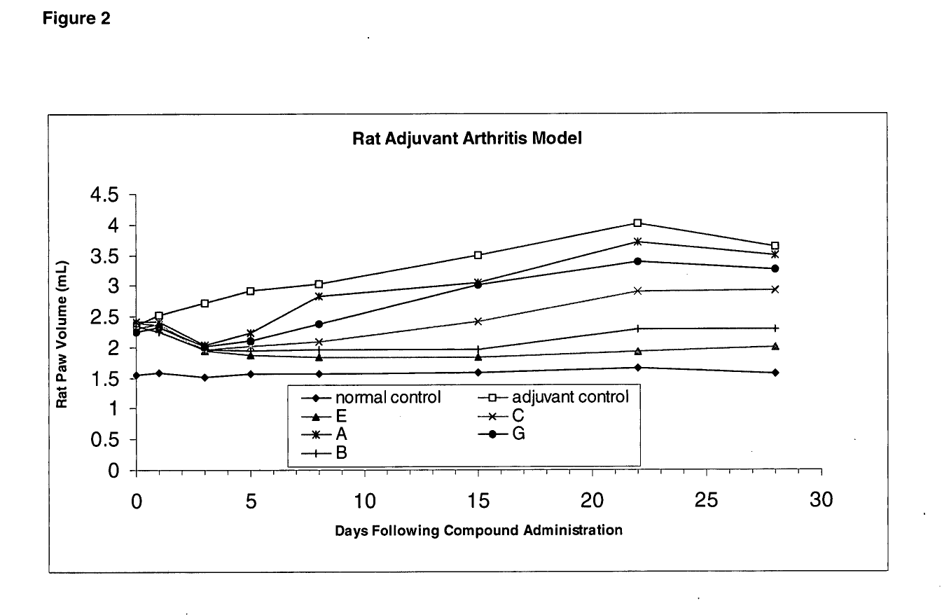

This example is summarized in FIG. 2 and shows the paw volume in rats plotted as a function of days following administration of the compounds to the rats. As seen from FIG. 2, compounds that minimized rat paw volume over longer time periods, such as compound E (4-[5-(3,4-dichlorophenyl)-3-(trifluoromethyl)-1H-pyrazol-1-yl]benzenesulfonamide), were considered effective extended duration compounds. All of the compounds tested in this model were effective for a minimum of 5 days.

### EXAMPLE 4

This example illustrates cassette-dosed pharmacokinetic tests in canines.

Cassette-dosing refers to the practice of dosing 2 or more compounds simultaneously to animals. This practice allows the pharmacokinetics of 2 or more compounds to be determined in a single experiment. The Cox-2 selective inhibitor compounds tested in this example were synthesized according to the methods described in, for example, U.S. Pat. No. 5,466,823 to Talley, et. al. In this experiment two or three of the halogen, haloalkyl, haloalkoxy, alkyl, nitrile, cyano or alkoxy substituted pyrazolyl benzenesulfonamides of the present invention were dissolved in an aqueous polyethylene glycol solution and orally administered once as a cassette by gastric gavage to canines (beagles) at a dosage of 2 mg/kg for each compound. Blood samples were collected over time intervals ranging from 0 to 50 days into EDTA tubes and plasma harvested. Plasma drug concentrations were then determined for each compound in the cassette by liquid chromatography-mass spectrometry methods. This process was repeated for each group of compounds (cassette) tested.

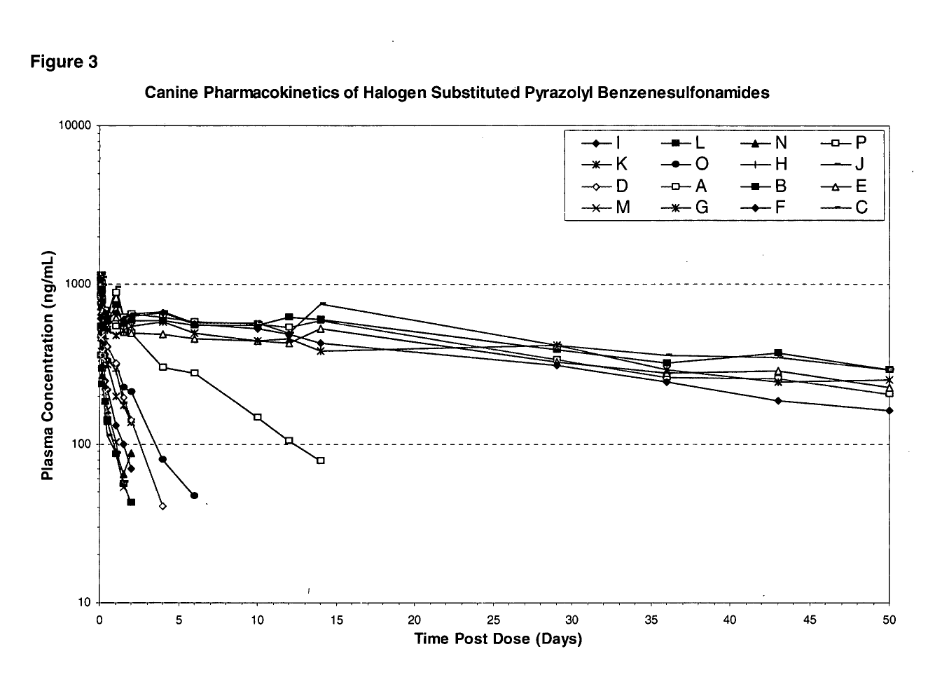

This example is summarized in FIG. 3 and shows a canine cassette-dosed pharmacokinetic study, where the mean plasma concentration in canines of the Cox-2 selective inhibitor (D-4-[5-phenyl-3-(trifluoromethyl)-1H-pyrazol-1-yl]benzenesulfonamide) is depicted as a function of time, along with the mean plasma concentration of Cox-2 selective inhibitors having extended duration properties according to the present invention, where all Cox-2 selective inhibitors were orally administered at 2 mg/kg. FIG. 3 shows that several of the extended duration compounds maintain high plasma concentrations in the canine model for at least 50 days after a single dose. The plasma half-lives (t_{1/2}) determined in this example are provided in Table 1.

### EXAMPLE 5

This example illustrates pharmacokinetics experiments performed to determine the plasma concentrations of extended duration Cox-2 selective inhibitor compounds in canines over time.

The Cox-2 selective inhibitor compounds tested in this example were synthesized according to the methods described in, for example, U.S. Pat. No. 5,466,823 to Talley, et. al. In this experiment, the halogen, haloalkyl, haloalkoxy, alkyl, nitrile, cyano or alkoxy substituted pyrazolyl benzenesulfonamides of the present invention were dissolved in an aqueous polyethylene glycol solution and orally administered once by gastric gavage to canines (beagles) at 2 or 4 mg/kg. In this example, each compound was administered individually to canines (not cassette dosed). Blood samples were collected over time intervals ranging from 0 to 768 hours after dosing. Plasma drug concentrations were then determined by liquid chromatography-mass spectrometry methods. This process was repeated for each compound tested.

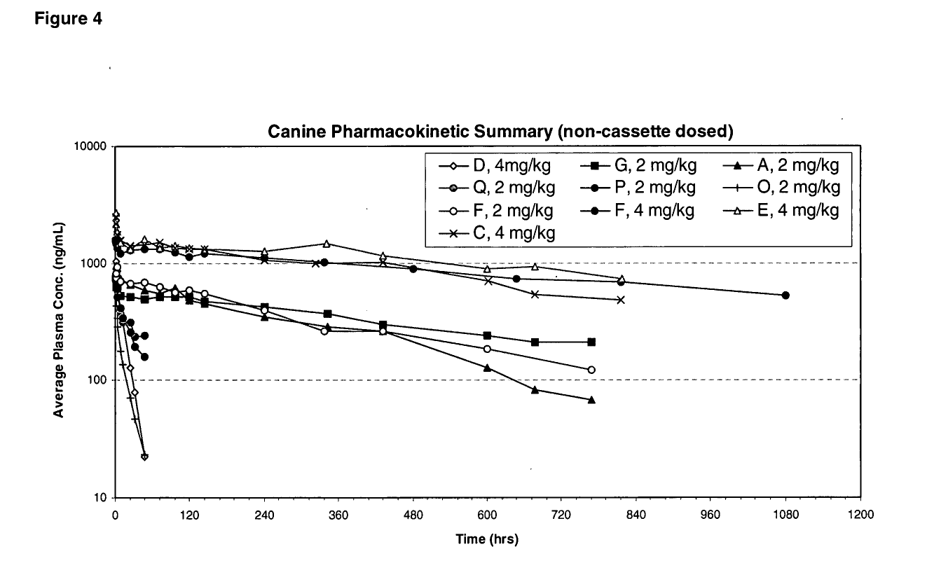

This example is summarized in FIG. 4 and shows the mean plasma concentration in canines of a Cox-2 selective inhibitor (D-4-[5-phenyl-3—(trifluoromethyl)-1H-pyrazol-1-yl]benzenesulfonamide) depicted as a function of time, along with the mean plasma concentrations of extended duration compounds of the present invention. FIG. 4 shows that several of the extended duration compounds described herein maintain high plasma concentrations for at least 768 hours (32 days). The plasma half-lives (t_{1/2}) determined in this example are provided in Table 1.

### EXAMPLE 6

This example illustrates experiments to test the effect of the extended duration compounds of the present invention on a canine acute pain efficacy model showing mean lameness scores over time.

In this example a canine model of acute pain was used to determine the efficacy and duration of Cox-2 selective inhibitor compounds. The canine acute pain model used was modified from the methods of Hargreaves, K. et al (Pain. 1988, 32:77-88) and Brooks, R. R. et al (J. Pharmacological Methods. 1991. 25:275-283). Briefly, the COX-2 compound being tested was orally administered once to canines (beagles) at a dose of 2 mg/kg or 4 mg/kg. At a predetermined time after dosing, carrageenan was injected into the canine paw. The amount of carrageenan injected was sufficient to produce a brief period of lameness in the canine. Clinical lameness was then evaluated by study personnel blinded to treatment using a subjective lameness scale from 0 (normal, non-lame) to 6 (non-weight bearing lameness). The time between the dose administration and the clinical lameness assessment was time point recorded to assess the duration of effect. In each experiment, the lameness of the Cox-2 inhibitor treated animals were compared to a control group of animals that did not receive drug but did receive carrageenan. The effectiveness of the Cox-2 inhibitors tested were also compared to a marketed NSAID for canines (Rimadyl®). The testing of Rimadyl treated animals was conducted using the same method with the exception that Rimadyl was administered at approximately 2.2 mg/kg twice daily for 4 consecutive days and lameness was assessed approximately 7-9 hours following the first dose on the fourth day of dosing.

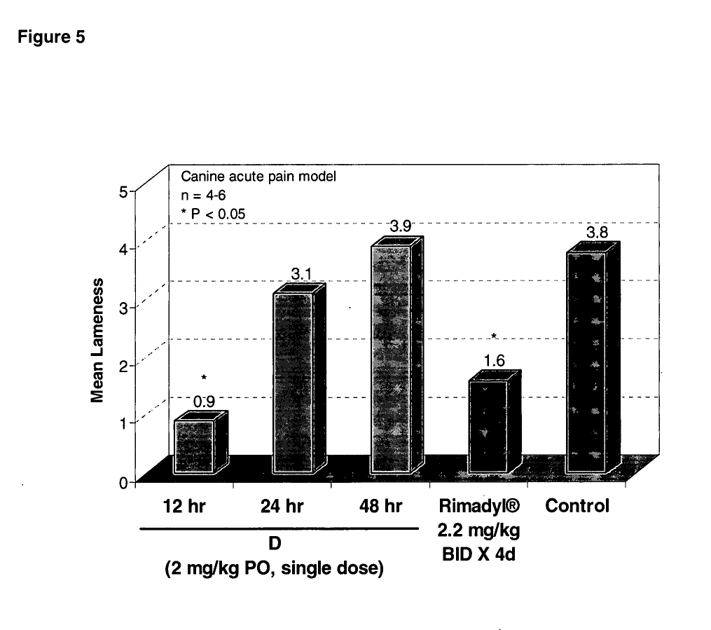

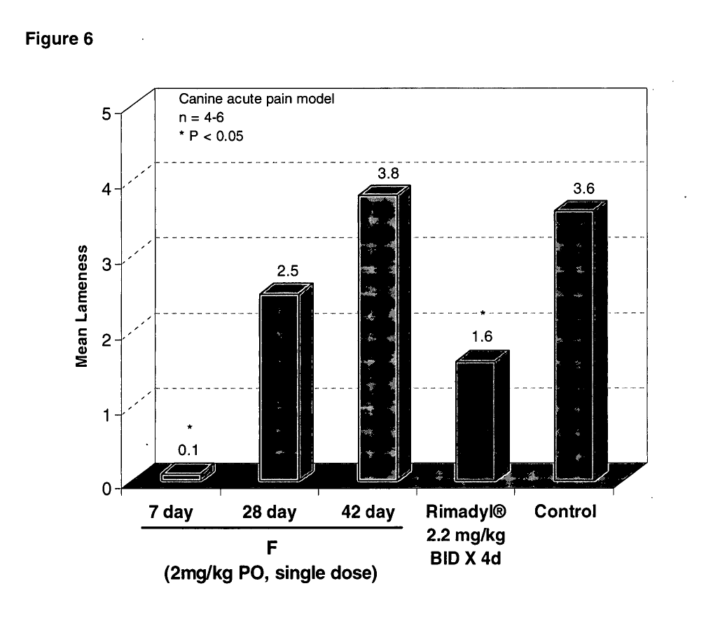

This example is summarized in FIGS. 5, 6 and 7 which shows graphs of the canine acute pain efficacy model plotting the mean lameness scores in canines after administration of 4-[5-phenyl-3-(trifluoromethyl)-1H-pyrazol-1-yl]benzenesulfonamide, 4-[5-(4-fluorophenyl)-3-(trifluoromethyl)-1H-pyrazol-1-yl]benzenesulfonamide, 4-[5-(2,4-[difluorophenyl)-3-(trifluoromethyl)-1H-pyrazol-1-yl]benzenesulfonamide, 4-[5-(2,4-[dichlorophenyl)-3-(trifluoromethyl)-1H-pyrazol-1-yl]benzenesulfonamide, and 4-[5-(3,4-[dichlorophenyl)-3-(trifluoromethyl)-1H-pyrazol-1-yl]benzenesulfonamide over time. The Cox-2 selective inhibitor compounds tested in this example were synthesized according to the methods described in, for example, U.S. Pat. No. 5,466,823 to Talley, et. al. For comparison, FIGS. 5 and 6 also show the mean lameness score for the control group and the Rimadyl® treated group.

In FIG. 5, the lameness of canines treated with a Cox-2 selective inhibitor (4-[5-phenyl-3-(trifluoromethyl)-1H-pyrazol-1-yl]benzenesulfonamide) was determined 12, 24 and 48 hours after a single 2 mg/kg oral dose. As seen in FIG. 5, 4-[5-phenyl-3-(trifluoromethyl)-1H-pyrazol-1-yl]benzenesulfonamide produced a significant reduction in lameness at 12 hours following administration, but not at 24 or 48 hours following administration. Therefore, a 2 mg/kg dose of 4-[5-phenyl-3-(trifluoromethyl)-1H-pyrazol-1-yl]benzenesulfonamide would need to be administered at least once per day to maintain a therapeutic response for an extended duration.

In FIG. 6, the lameness of canines treated with an extended duration Cox-2 selective inhibitor of the present invention (4-[5-(4-fluorophenyl)-3-(trifluoromethyl)-1H-pyrazol-1-yl]benzenesulfonamide) was determined at 7, 28 and 42 days following a single 2 mg/kg oral dose. Administration of 4-[5-(4-fluorophenyl)-3-(trifluoromethyl)-1H-pyrazol-1-yl]benzenesulfonamide resulted in a significant reduction in lameness for at least 7 days following a single dose.

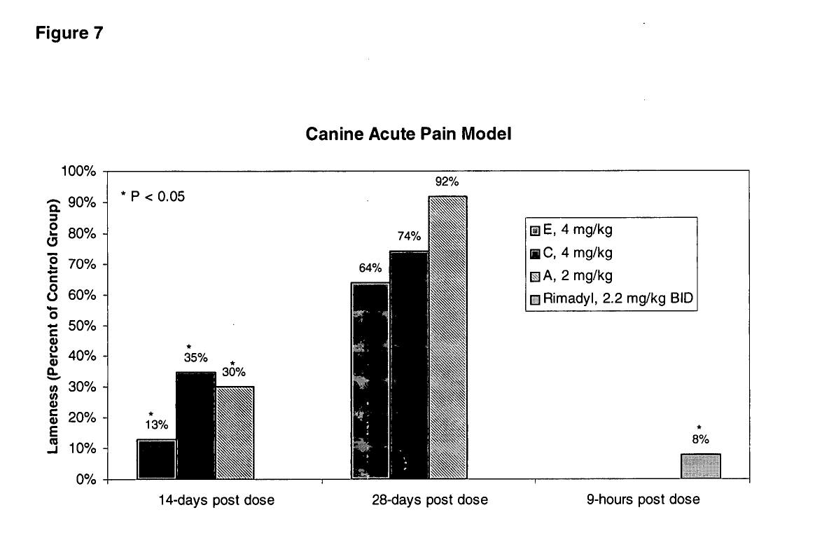

In FIG. 7, the lameness of canines (expressed as a percent of the control group) treated with a extended duration Cox-2 selective inhibitors of the present invention (4-[5-(2,4-[difluorophenyl)-3-(trifluoromethyl)-1H-pyrazol-1-yl]benzenesulfonamide, 4-[5-(2,4-[dichlorophenyl)-3-(trifluoromethyl)-1H-pyrazol-1-yl]benzenesulfonamide, and 4-[5-(3,4-[dichlorophenyl)-3-(trifluoromethyl)-1H-pyrazol-1-yl]benzenesulfonamide) was determined at 14 and 28 days following a single 2 mg/kg or 4 mg/kg oral dose. As seen in FIG. 7, a single administration of 4-[5-(2,4-[difluorophenyl)-3-(trifluoromethyl)-1H-pyrazol-1-yl]benzenesulfonamide at 2 mg/kg, 4-[5-(2,4-[dichlorophenyl)-3-(trifluoromethyl)-1H-pyrazol-1-yl]benzenesulfonamide at 4 mg/kg, and 4-[5-(3,4-[dichlorophenyl)-3-(trifluoromethyl)-1H-pyrazol-1-yl]benzenesulfonamide at 4 mg/kg resulted in a significant reduction in lameness for at least 14 days.

### EXAMPLE 7

This example shows the general preparation of celecoxib and therefore, a general method of preparation for various halogen, haloalkyl, haloalkoxy, alkyl, nitrile, cyano or alkoxy substituted diaryl-substituted pyrazolyl Cox-2 selective inhibitors.

Step 1: Preparation of 1-(4-methylphenyl)-4,4,4-trifluorobutane-1,3-dione.

Following the disclosure provided in U.S. Pat. No. 5,760,068, 4′-Methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCl was added and the mixture extracted with 4×75 mL ethyl acetate. The extracts were dried over MgSO₄, filtered and concentrated to afford 8.47 g (94%) of a brown oil which was carried on without further purification.

Step 2: Preparation of 4-[5-(4-methylphenyl)-3-(trifluoromethyl)-1H-pyrazol-1-yl]benzenesulfonamide.

To the dione from Step 1 (4.14 g, 18.0 mmol) in 75 mL absolute ethanol, 4.26 g (19.0 mmol) 4-sulphonamidophenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157°-159° C.; and a calculated composition of C₁₇H₁₄N₃O₂SF₃; C, 53.54; H, 3.70; N, 11.02. The composition that was found by analysis was: C, 53.17; H, 3.81; N, 10.90.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description and shown in the accompanying drawings shall be interpreted as illustrative and not in a limiting sense. These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the preferred versions contained therein. In addition, it should be understood that aspects of the various embodiments may be interchanged both in whole or in part.

1. A method of providing extended duration prevention and treatment of pain, inflammation and inflammation-related disorders in a subject in need of such extended duration treatment or prevention, the method comprising administering to the subject a compound having the formula: 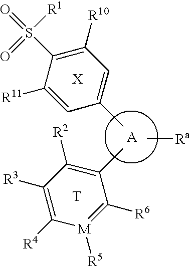
wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen; and
including the diastereomers, enantiomers, racemates, tautomers, salts, esters, amides and prodrugs thereof.

2. The method according to claim 1, wherein:
R² and R⁶ are each independently selected from the group consisting of hydrogen and halo;
R³ and R⁵ are each independently selected from the group consisting of hydrogen, halo, alkyl and alkoxy;
R⁴ is selected from the group consisting of halo, alkyl and alkoxy, except that when R⁴ is alkoxy, R³ is other than fluoro, when M is nitrogen, R⁴ is other than methyl, and when R⁴ is methyl, one of R², R³, R⁵ and R⁶ is other than hydrogen; and
R¹⁰ and R¹¹ are each independently selected from the group consisting of hydrogen and halo.

3. The method according to claim 1, wherein the extended duration cycloxygenase-2 selective inhibitor comprises a compound having the formula: 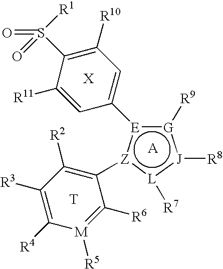
wherein:
E is selected from the group consisting of nitrogen, sulfur and carbon;
G is selected from the group consisting of nitrogen, sulfur, oxygen and carbon;
J is selected from the group consisting of nitrogen, sulfur, carbon and oxygen;
L is selected from the group consisting of nitrogen, oxygen and carbon;
Z is selected from the group consisting of nitrogen and carbon;
M is selected from the group consisting of nitrogen and carbon;
R¹ is selected from the group consisting of hydrogen, propanamide, amino and methyl;
R², R³, R⁵, R⁶, R¹⁰ and R¹¹ are optionally present and independently selected from the group consisting of hydrogen, cyano, nitrile, methyl, methoxy, fluorine, chlorine, bromine, iodine, astatine and haloalkyl;
R⁴ is optionally present and independently selected from the group consisting of hydrogen, cyano, nitrile, methyl, methoxy, fluorine, chlorine, bromine, iodine, astatine, haloalkyl and substituted or unsubstituted heterocycle, except that when R⁴ is methoxy, R³ is other than fluoro, when M is nitrogen, R⁴ is other than methyl, and when R⁴ is methyl, one of R², R³, R⁵ and R⁶ is other than hydrogen;
R⁷ is optionally present and is independently selected from the group consisting of hydrogen and oxygen;
R⁸ is optionally present and is independently selected from the group consisting of hydrogen, alkyl and haloalkyl;
R⁹ is optionally present and is independently selected from the group consisting of hydrogen, alkyl and haloalkyl; and
including diastereomers, enantiomers, racemates, tautomers, salts, esters, amides and prodrugs thereof.

4. The method according to claim 1, wherein the extended duration cycloxygenase-2 selective inhibitor comprises a compound having the formula: 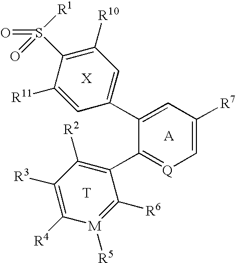
wherein:
M is selected from the group consisting of nitrogen and carbon;
Q is selected from the group consisting of nitrogen and carbon;
R¹ is selected from the group consisting of hydrogen, propanamide, and methyl;
R², R³, R⁵, R⁶, R¹⁰ and R¹¹ are optionally present and independently selected from the group consisting of hydrogen, cyano, nitrile, methyl, methoxy, fluorine, chlorine, bromine, iodine, astatine and haloalkyl;
R⁴ is optionally present and independently selected from the group consisting of hydrogen, cyano, nitrile, methyl, methoxy, fluorine, chlorine, bromine, iodine, astatine, haloalkyl and substituted or unsubstituted heterocycle, except that when R⁴ is methoxy, R³ is other than fluoro, when M is nitrogen, R⁴ is other than methyl, and when R⁴ is methyl, one of R², R³, R⁵ and R⁶ is other than hydrogen;
R⁷ is selected from the group consisting of hydrogen, chlorine, fluorine, bromine, iodine and astatine; and
including diastereomers, enantiomers, racemates, tautomers, salts, esters, amides and prodrugs thereof.

5. The method according to claim 2, wherein:
R² and R⁶ are each independently selected from the group consisting of hydrogen and halo;
R³ and R⁵ are each independently selected from the group consisting of hydrogen, halo, alkyl and alkoxy;
R⁴ is selected from the group consisting of halo and alkoxy, except that when R⁴ is alkoxy, R³ is other than fluoro;
R¹⁰ and R¹¹ are each independently selected from the group consisting of hydrogen and halo;

6. The method according to claim 5, wherein:
R² and R⁶ are each independently selected from the group consisting of hydrogen, chlorine, bromine, iodine and fluorine;
R³ and R⁵ are each independently selected from the group consisting of hydrogen, chlorine, bromine, fluorine, methyl and methoxy;
R⁴ is selected from the group consisting of chlorine, bromine, iodine, fluorine and methoxy, except that when R⁴ is methoxy, R³ is other than fluoro; and
R¹⁰ and R¹¹ are each independently selected from the group consisting of hydrogen, chlorine, bromine, iodine and fluorine.

7. The method according to claim 2, wherein:
R² and R⁶ are each independently selected from the group consisting of hydrogen and halo;
R³ and R⁵ are each independently selected from the group consisting of hydrogen, halo, alkyl and alkoxy;
R⁴ is halo; and
R¹⁰ and R¹¹ are each independently selected from the group consisting of hydrogen and halo.

8. The method according to claim 7, wherein:
R² and R⁶ are each independently selected from the group consisting of hydrogen, chlorine, bromine, iodine and fluorine;
R³ and R⁵ are each independently selected from the group consisting of hydrogen, chlorine, bromine, iodine, fluorine, methyl and methoxy;
R⁴ is selected from the group consisting of chlorine, iodine, fluorine and bromine; and
R¹⁰ and R¹¹ are each independently selected from the group consisting of hydrogen, chlorine, bromine, iodine and fluorine.

9. The method according to claim 2, wherein:
R² and R⁶ are each independently selected from the group consisting of hydrogen and halo;
R³ and R⁵ are each independently selected from the group consisting of hydrogen, halo and alkyl;
R⁴ is halo; and
R¹⁰ and R¹¹ are each independently selected from the group consisting of hydrogen and halo.

10. The method according to claim 9, wherein:
R² and R⁶ are each independently selected from the group consisting of hydrogen, chlorine, fluorine, iodine and bromine;
R³ and R⁵ are each independently selected from the group consisting of hydrogen, chlorine, fluorine, iodine, bromine and methyl;
R⁴ is selected from the group consisting of chlorine, fluorine, iodine and bromine; and
R¹⁰ and R¹¹ are each independently selected from the group consisting of hydrogen, chlorine, bromine, iodine and fluorine.

11. The method according to claim 2, wherein:
R² and R⁶ are each independently selected from the group consisting of hydrogen and halo;
R³ and R⁵ are each independently selected from the group consisting of hydrogen and halo; and
R⁴ is halo.

12. The method according to claim 2, wherein:
R² and R⁶ are each independently selected from the group consisting of hydrogen, chlorine, fluorine, iodine and bromine;
R³ and R⁵ are each independently selected from the group consisting of hydrogen, chlorine, fluorine, iodine and bromine;
R⁴ is selected from the group consisting of chlorine, fluorine, iodine and bromine; and
R¹⁰ and R¹¹ are each independently selected from the group consisting of hydrogen, chlorine, bromine, iodine and fluorine.

13. The method according to claim 2, wherein:
R² and R⁶ are each hydrogen;
R³ and R⁵ are each independently selected from the group consisting of hydrogen and halo;
R⁴ is halo; and
R¹⁰ and R¹¹ are each hydrogen.

14. The method according to claim 2, wherein:
R² and R⁶ are each hydrogen;
R³ and R⁵ are each independently selected from the group consisting of hydrogen, chlorine, fluorine, iodine and bromine; and
R⁴ is selected from the group consisting of chlorine, fluorine, iodine and bromine.
R¹⁰ and R¹¹ are each hydrogen.

15. The method according to claim 2, wherein:
R⁴ is halo; and
R², R³, R⁵, R⁶, R¹⁰ and R¹¹ are each hydrogen.

16. The method according to claim 15, wherein:
R⁴ is selected from the group consisting of chlorine, fluorine, bromine and iodine; and
R², R³, R⁵, R⁶, R¹⁰ and R¹¹ are each hydrogen.

17. The method according to claim 16, wherein:
R⁴ is selected from the group consisting of chlorine and fluorine.

18. The method according to claim 17, wherein:
R⁴ is chlorine.

19. The method according to claim 1, wherein the plasma half-life of the extended duration Cox-2 selective inhibitor in a rat model assay is from about 24 hours to about 1176 hours.

20. The method according to claim 19, wherein the plasma half-life of the extended duration Cox-2 selective inhibitor in a rat model assay is from about 24 hours to about 336 hours.

21. The method according to claim 20, wherein the plasma half-life of the extended duration Cox-2 selective inhibitor in a rat model assay is from about 48 hours to about 192 hours.

22. The method according to claim 1, wherein the plasma half-life of the extended duration Cox-2 selective inhibitor in a rat model assay is at least 24 hours.

23. The method according to claim 22, wherein the plasma half-life of the extended duration Cox-2 selective inhibitor in a rat model assay is at least 36 hours.

24. The method according to claim 23, wherein the plasma half-life of the extended duration Cox-2 selective inhibitor in a rat model assay is at least 48 hours.

25. The method according to claim 24, wherein the plasma half-life of the extended duration Cox-2 selective inhibitor in a rat model assay is at least 72 hours.

26. The method according to claim 1, wherein the extended duration Cox-2 selective inhibitor has a selectivity for Cox-2 over Cox-1 of at least about 5.

27. The method according to claim 26, wherein the extended duration Cox-2 selective inhibitor has a selectivity for Cox-2 over Cox-1 of at least about 50.

28. The method according to claim 27, wherein the extended duration Cox-2 selective inhibitor has a selectivity for Cox-2 over Cox-1 of at least about 100.

29. The method according to claim 28, wherein the extended duration Cox-2 selective inhibitor has a selectivity for Cox-2 over Cox-1 of at least about 300.

30. The method according to claim 29, wherein the extended duration Cox-2 selective inhibitor has a selectivity for Cox-2 over Cox-1 of at least about 500.

31. The method according to claim 1, wherein the extended duration Cox-2 selective inhibitor is selected from the group consisting of 4-[5-(2,4-[difluorophenyl)-3-(trifluoromethyl)-1H-pyrazol-1-yl]benzenesulfonamide, 4-[5-(4-chlorophenyl)-3-(trifluoromethyl)-1H-pyrazol-1-yl]benzenesulfonamide, 4-[5-(2,4-[dichlorophenyl)-3-(trifluoromethyl)-1H-pyrazol-1-yl]benzenesulfonamide, 4-[5-(3,4-dichlorophenyl)-3-(trifluoromethyl)-1H-pyrazol-1-yl]benzenesulfonamide, 4-[5-(4-fluorophenyl)-3-(trifluoromethyl)-1H-pyrazol-1-yl]benzenesulfonamide, 4-[5-(3,4-difluorophenyl)-3-(trifluoromethyl)-1H-pyrazol-1-yl]benzenesulfonamide, 4-[5-(4-methoxyphenyl)-3-(trifluoromethyl)-1H-pyrazol-1-yl]benzenesulfonamide, 4-[5-(3-chloro-4-methoxyphenyl)-3-(trifluoromethyl)-1H-pyrazol-1-yl]benzenesulfonamide, 4-[5-(3,5-dichloro-4-methoxyphenyl)-3-(trifluoromethyl)-1H-pyrazol-1-yl]benzenesulfonamide, 4-[5-(4-methoxy-3-methylphenyl)-3(trifluoromethyl)-1H-pyrazol-1-yl]benzenesulfonamide, 4-[5-(4-methoxy-3,5-dimethylphenyl)-3-(trifluoromethyl)-1H-pyrazol-1-yl]benzenesulfonamide, 4-[5-(3-chloro-4-methylphenyl)-3-(trifluoromethyl)-1H-pyrazol-1-yl]benzenesulfonamide, 4-[5-(3,5-dichloro-4-methylphenyl)-3-(trifluoromethyl)-1H-pyrazol-1-yl]benzenesulfonamide, 4-[5-(4-chloro-3-methylphenyl)-3-(trifluoromethyl)-1H-pyrazol-1-yl]benzenesulfonamide, 4-[5-(4-chloro-3-methoxyphenyl)-3-(trifluoromethyl)-1H-pyrazol-1-yl]benzenesulfonamide, 4-[5-(4-bromo-3-methoxyphenyl)-3-(trifluoromethyl)-1H-pyrazol-1-yl]benzenesulfonamide, and 4-[3-(4-chlorophenyl)-5-(trifluoromethyl)isoxazol-4-yl]benzenesulfonamide.

32. The method according to claim 1, wherein the extended duration Cox-2 selective inhibitor is selected from the group consisting of 4-[5-(4-chlorophenyl)-3-(trifluoromethyl)-1H-pyrazol-1-yl]benzenesulfonamide, 4-[5-(3,4-dichlorophenyl)-3-(trifluoromethyl)-1H-pyrazol-1-yl]benzenesulfonamide, 4-[5-(2,4-[dichlorophenyl)-3-(trifluoromethyl)-1H-pyrazol-1-yl]benzenesulfonamide, 4-[5-(4-fluorophenyl)-3-(trifluoromethyl)-1H-pyrazol-1-yl]benzenesulfonamide, 4-[5-(3,4-difluorophenyl)-3-(trifluoromethyl)-1H-pyrazol-1-yl]benzenesulfonamide, and 4-[5-(2,4-[difluorophenyl)-3-(trifluoromethyl)-1H-pyrazol-1-yl]benzenesulfonamide.

33. The method according to claim 1, wherein the extended duration Cox-2 selective inhibitor comprises 4-[5-(4-chlorophenyl)-3-(trifluoromethyl)-1H-pyrazol-1-yl]benzenesulfonamide.

34. The method according to claim 1, wherein the haloalkyl comprises a compound having fluorine, chlorine, bromine, iodine, or astatine covalently coupled with an alkyl, alkenyl, alkynyl, alkoxy, aralkyl, aryl, carbonyl, cycloalkyl, benzyl, phenyl, alicyclic or heterocyclic group.

35. The method according to claim 1, wherein the subject is in need of the treatment or prevention of a disorder selected from the group consisting of connective tissue and joint disorders, neoplasia disorders, cardiovascular disorders, otic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecology and obstetric disorders, injury and trauma disorders, surgical disorders, dental and oral disorders, dermatologic disorders, hematological disorders, poisoning disorders and any other disorder that involves any type of inflammation-related process.

36. The method according to claim 35, wherein the subject is in need of the treatment or prevention of a connective tissue and joint disorder.

37. A method of increasing the plasma half-life of a Cox-2 selective inhibitor comprising adding one or more substituent groups onto either or both of the “T” ring and “X” ring of a Cox-2 selective inhibitor having the structure: 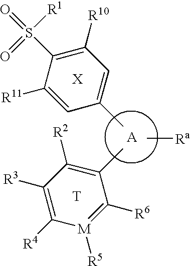
to provide an extended duration Cox-2 selective inhibitor wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen, haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen; and
including the diastereomers, enantiomers, racemates, tautomers, salts, esters, amides and prodrugs thereof.

38. The method according to claim 37, wherein:
R² and R⁶ are each independently selected from the group consisting of hydrogen and halo;
R³ and R⁵ are each independently selected from the group consisting of hydrogen, halo, alkyl and alkoxy;
R⁴ is selected from the group consisting of halo, alkyl and alkoxy, except that when R⁴ is alkoxy, R³ is other than fluoro, when M is nitrogen, R⁵ is absent, and when R⁴ is methyl, one of R², R³, R⁵ and R⁶ is other than hydrogen; and
R¹⁰ and R¹¹ are each independently selected from the group consisting of hydrogen and halo.

39. The method according to claim 37, wherein the extended duration cycloxygenase-2 selective inhibitor comprises a compound having the formula: 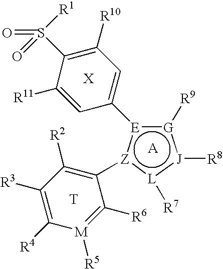
wherein:
E is selected from the group consisting of nitrogen, sulfur and carbon;
G is selected from the group consisting of nitrogen, sulfur, oxygen and carbon;
J is selected from the group consisting of nitrogen, sulfur, carbon and oxygen;
L is selected from the group consisting of nitrogen, oxygen and carbon;
Z is selected from the group consisting of nitrogen and carbon;
M is selected from the group consisting of nitrogen and carbon;
R¹ is selected from the group consisting of hydrogen, propanamide, amino and methyl;
R², R³, R⁵, R⁶, R¹⁰ and R¹¹ are optionally present and independently selected from the group consisting of hydrogen, cyano, nitrile, methyl, methoxy, fluorine, chlorine, bromine, iodine, astatine and haloalkyl;
R⁴ is optionally present and independently selected from the group consisting of hydrogen, cyano, nitrile, methyl, methoxy, fluorine, chlorine, bromine, iodine, astatine, haloalkyl and substituted or unsubstituted heterocycle, except that when R⁴ is methoxy, R³ is other than fluoro, when M is nitrogen, R⁴ is other than methyl, and when R⁴ is methyl, one of R², R³, R⁵ and R⁶ is other than hydrogen;
R⁷ is optionally present and is independently selected from the group consisting of hydrogen and oxygen;
R⁸ is optionally present and is independently selected from the group consisting of hydrogen, alkyl and haloalkyl;
R⁹ is optionally present and is independently selected from the group consisting of hydrogen, alkyl and haloalkyl; and
including diastereomers, enantiomers, racemates, tautomers, salts, esters, amides and prodrugs thereof.

40. The method according to claim 37, wherein the extended duration cycloxygenase-2 selective inhibitor comprises a compound having the formula: 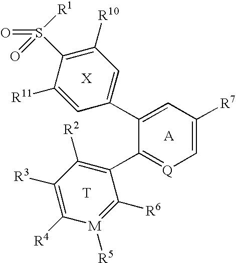
wherein:
M is selected from the group consisting of nitrogen and carbon;
Q is selected from the group consisting of nitrogen and carbon;
R¹ is selected from the group consisting of hydrogen, propanamide, and methyl;
R², R³, R⁵, R⁶, R¹⁰ and R¹¹ are optionally present and independently selected from the group consisting of hydrogen, cyano, nitrile, methyl, methoxy, fluorine, chlorine, bromine, iodine, astatine and haloalkyl;
R⁴ is optionally present and independently selected from the group consisting of hydrogen, cyano, nitrile, methyl, methoxy, fluorine, chlorine, bromine, iodine, astatine, haloalkyl and substituted or unsubstituted heterocycle, except that when R⁴ is methoxy, R³ is other than fluoro, when M is nitrogen, R⁴ is other than methyl, and when R⁴ is methyl, one of R², R³, R⁵ and R⁶ is other than hydrogen;
R⁷ is selected from the group consisting of hydrogen, chlorine, fluorine, bromine, iodine and astatine; and
including diastereomers, enantiomers, racemates, tautomers, salts, esters, amides and prodrugs thereof.

41. The method according to claim 37, wherein:
R² and R⁶ are each independently selected from the group consisting of hydrogen and halo;
R³ and R⁵ are each independently selected from the group consisting of hydrogen, halo, alkyl and alkoxy;
R⁴ is selected from the group consisting of halo and alkoxy, except that when R⁴ is alkoxy, R³ is other than fluoro; and
R¹⁰ and R¹¹ are each independently selected from the group consisting of hydrogen and halo.

42. The method according to claim 41, wherein:
R² and R⁶ are each independently selected from the group consisting of hydrogen, chlorine, bromine, iodine and fluorine;
R³ and R⁵ are each independently selected from the group consisting of hydrogen, chlorine, bromine, fluorine, methyl and methoxy;
R⁴ is selected from the group consisting of chlorine, bromine, iodine, fluorine and methoxy, except that when R⁴ is methoxy, R³ is other than fluoro; and
R¹⁰ and R¹¹ are each independently selected from the group consisting of hydrogen, chlorine, bromine, iodine and fluorine.

43. The method according to claim 38, wherein:
R² and R⁶ are each independently selected from the group consisting of hydrogen and halo;
R³ and R⁵ are each independently selected from the group consisting of hydrogen, halo, alkyl and alkoxy;
R⁴ is halo; and
R¹⁰ and R¹¹ are each independently selected from the group consisting of hydrogen and halo.

44. The method according to claim 43, wherein:
R² and R⁶ are each independently selected from the group consisting of hydrogen, chlorine, bromine, iodine and fluorine;
R³ and R⁵ are each independently selected from the group consisting of hydrogen, chlorine, bromine, iodine, fluorine, methyl and methoxy;
R⁴ is selected from the group consisting of chlorine, iodine, fluorine and bromine; and
R¹⁰ and R¹¹ are each independently selected from the group consisting of hydrogen, chlorine, bromine, iodine and fluorine.

45. The method according to claim 38, wherein:
R² and R⁶ are each independently selected from the group consisting of hydrogen and halo;
R³ and R⁵ are each independently selected from the group consisting of hydrogen, halo and alkyl;
R⁴ is halo; and
R¹⁰ and R¹¹ are each independently selected from the group consisting of hydrogen and halo.

46. The method according to claim 45, wherein:
R² and R⁶ are each independently selected from the group consisting of hydrogen, chlorine, fluorine, iodine and bromine;
R³ and R⁵ are each independently selected from the group consisting of hydrogen, chlorine, fluorine, iodine, bromine and methyl;
R⁴ is selected from the group consisting of chlorine, fluorine, iodine and bromine; and
R¹⁰ and R¹¹ are each independently selected from the group consisting of hydrogen, chlorine, bromine, iodine and fluorine.

47. The method according to claim 38, wherein:
R² and R⁶ are each independently selected from the group consisting of hydrogen and halo;
R³ and R⁵ are each independently selected from the group consisting of hydrogen and halo;
R⁴ is halo; and
R¹⁰ and R¹¹ are each independently selected from the group consisting of hydrogen and halo.

48. The method according to claim 47, wherein:
R² and R⁶ are each independently selected from the group consisting of hydrogen, chlorine, fluorine, iodine and bromine;
R³ and R⁵ are each independently selected from the group consisting of hydrogen, chlorine, fluorine, iodine and bromine; and
R⁴ is selected from the group consisting of chlorine, fluorine, iodine and bromine; and
R¹⁰ and R¹¹ are each independently selected from the group consisting of hydrogen, chlorine, bromine, iodine and fluorine.

49. The method according to claim 38, wherein:
R², R⁶, R¹⁰ and R¹¹ are each hydrogen;
R³ and R⁵ are each independently selected from the group consisting of hydrogen and halo;
R⁴ is halo.

50. The method according to claim 49, wherein:
R², R⁶, R¹⁰ and R¹¹ are each hydrogen;
R³ and R⁵ are each independently selected from the group consisting of hydrogen, chlorine, fluorine, iodine and bromine; and
R⁴ is selected from the group consisting of chlorine, fluorine, iodine and bromine.

51. The method according to claim 38, wherein:
R⁴ is halo; and
R², R³, R⁵, R⁶, R¹⁰ and R¹¹ are each hydrogen.

52. The method according to claim 51, wherein:
R⁴ is selected from the group consisting of chlorine, fluorine, bromine and iodine; and
R², R³, R⁵, R⁶, R¹⁰ and R¹¹ are each hydrogen.

53. The method according to claim 53, wherein:
R⁴ is selected from the group consisting of chlorine and fluorine.

54. The method according to claim 53, wherein:
R⁴ is chlorine.

55. The method according to claim 37, wherein the plasma half-life of the extended duration Cox-2 selective inhibitor in a rat model assay is from about 24 hours to about 1176 hours.

56. The method according to claim 55, wherein the plasma half-life of the extended duration Cox-2 selective inhibitor in a rat model assay is from about 36 hours to about 336 hours.

57. The method according to claim 56, wherein the plasma half-life of the extended duration Cox-2 selective inhibitor in a rat model assay is from about 48 hours to about 192 hours.

58. The method according to claim 37, wherein the plasma half-life of the extended duration Cox-2 selective inhibitor in a rat model assay is at least 24 hours.

59. The method according to claim 58, wherein the plasma half-life of the extended duration Cox-2 selective inhibitor in a rat model assay is at least 24 hours.

60. The method according to claim 59, wherein the plasma half-life of the extended duration Cox-2 selective inhibitor in a rat model assay is at least 48 hours.

61. The method according to claim 60, wherein the plasma half-life of the extended duration Cox-2 selective inhibitor in a rat model assay is at least 72 hours.

62. The method according to claim 37, wherein the extended duration Cox-2 selective inhibitor has a selectivity for Cox-2 over Cox-1 of at least about 5.

63. The method according to claim 62, wherein the extended duration Cox-2 selective inhibitor has a selectivity for Cox-2 over Cox-1 of at least about 50.

64. The method according to claim 63, wherein the extended duration Cox-2 selective inhibitor has a selectivity for Cox-2 over Cox-1 of at least about 100.

65. The method according to claim 64, wherein the extended duration Cox-2 selective inhibitor has a selectivity for Cox-2 over Cox-1 of at least about 300.

66. The method according to claim 65, wherein the extended duration Cox-2 selective inhibitor has a selectivity for Cox-2 over Cox-1 of at least about 500.

67. The method according to claim 37, wherein the extended duration Cox-2 selective inhibitor is selected from the group consisting of 4-[5-(2,4-[difluorophenyl)-3-(trifluoromethyl)-1H-pyrazol-1-yl]benzenesulfonamide, 4-[5-(4-chlorophenyl)-3-(trifluoromethyl)-1H-pyrazol-1-yl]benzenesulfonamide, 4-[5-(2,4-[dichlorophenyl)-3-(trifluoromethyl)-1H-pyrazol-1-yl]benzenesulfonamide, 4-[5-(3,4-dichlorophenyl)-3-(trifluoromethyl)-1H-pyrazol-1-yl]benzenesulfonamide, 4-[5-(4-fluorophenyl)-3-(trifluoromethyl)-1H-pyrazol-1-yl]benzenesulfonamide, 4-[5-(3,4-difluorophenyl)-3-(trifluoromethyl)-1H-pyrazol-1-yl]benzenesulfonamide, 4-[5-(4-methoxyphenyl)-3-(trifluoromethyl)-1H-pyrazol-1-yl]benzenesulfonamide, 4-[5-(3-chloro-4methoxyphenyl)-3-(trifluoromethyl)-1H-pyrazol-1-yl]benzenesulfonamide, 4-[5-(3,5-dichloro-4-methoxyphenyl)-3-(trifluoromethyl)-1H-pyrazol-1-yl]benzenesulfonamide, 4-[5-(4-methoxy-3-methylphenyl)-3-(trifluoromethyl)-1H-pyrazol-1-yl]benzenesulfonamide, 4-[5-(4-methoxy-3,5-dimethylphenyl)-3-(trifluoromethyl)-1H-pyrazol-1-yl]benzenesulfonamide, 4-[5-(3-chloro-4-methylphenyl)-3-(trifluoromethyl)-1H-pyrazol-1-yl]benzenesulfonamide, 4-[5-(3,5-dichloro-4-methylphenyl)-3-(trifluoromethyl)-1H-pyrazol-1-yl]benzenesulfonamide, 4-[5-(4-chloro-3-methylphenyl)-3-(trifluoromethyl)-1H-pyrazol-1-yl]benzenesulfonamide, 4-[5-(4-chloro-3-methoxyphenyl)-3-(trifluoromethyl)-1H-pyrazol-1-yl]benzenesulfonamide, 4-[5-(4-bromo-3-methoxyphenyl)-3-(trifluoromethyl)-1H-pyrazol-1-yl]benzenesulfonamide, and 4-[3-(4-chlorophenyl)-5-(trifluoromethyl)isoxazol-4-yl]benzenesulfonamide.

68. The method according to claim 37, wherein the extended duration Cox-2 selective inhibitor is selected from the group consisting of 4-[5-(4-chlorophenyl)-3-(trifluoromethyl)-1H-pyrazol-1-yl]benzenesulfonamide, 4-[5-(3,4-dichlorophenyl)-3-(trifluoromethyl)-1H-pyrazol-1-yl]benzenesulfonamide, 4-[5-(2,4-[dichlorophenyl)-3-(trifluoromethyl)-1H-pyrazol-1-yl]benzenesulfonamide, 4-[5-(4-fluorophenyl)-3-(trifluoromethyl)-1H-pyrazol-1-yl]benzenesulfonamide, 4-[5-(3,4-difluorophenyl)-3-(trifluoromethyl)-1H-pyrazol-1-yl]benzenesulfonamide, and 4-[5-(2,4-[difluorophenyl)-3-(trifluoromethyl)-1H-pyrazol-1-yl]benzenesulfonamide.

69. The method according to claim 37, wherein the extended duration Cox-2 selective inhibitor comprises 4-[5-(4-chlorophenyl)-3-(trifluoromethyl)-1H-pyrazol-1-yl]benzenesulfonamide.

70. The method according to claim 37, wherein the haloalkyl comprises a compound having fluorine, chlorine, bromine, iodine, or astatine covalently coupled with an alkyl, alkenyl, alkynyl, alkoxy, aralkyl, aryl, carbonyl, cycloalkyl, benzyl, phenyl, alicyclic or heterocyclic group.

71. A method of reducing the dosing frequency of a diaryl-substituted Cox-2 selective inhibitor compound comprising:
a. adding one or more substituent groups onto either or both of the “T” ring and “X” ring of a Cox-2 selective inhibitor having the structure: 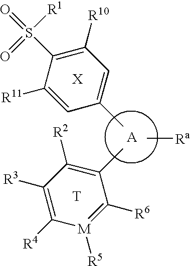
to provide an extended duration Cox-2 selective inhibitor wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen;
including the diastereomers, enantiomers, racemates, tautomers, salts, esters, amides and prodrugs thereof; and
b. administering a therapeutic amount of the resulting compound to a subject in need of such reduced frequency dosing.

72. A therapeutic composition comprising a compound having the structure: 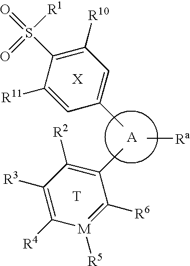
wherein:
A is an optionally substituted 5 membered ring or 6 membered ring;
R^{a} is optionally present, and if present is selected from the group consisting of halo, alkyl, haloalkyl and oxo;
M is selected from the group consisting of nitrogen and carbon;
R¹ is selected from the group consisting of hydrogen, propanamide, amino and methyl;
R², R³, R⁵, R⁶, R¹⁰ and R¹¹ are each independently selected from the group consisting of hydrogen, halogen, haloalkyl, haloalkoxy, alkyl, nitrile, cyano and alkoxy;
R⁴ is independently selected from the group consisting of hydrogen, halogen haloalkyl, haloalkoxy, alkyl, nitrile, cyano, alkoxy and substituted or unsubstituted heterocycle;
at least one of R², R³, R⁴, R⁵, R⁶, R¹⁰ and R¹¹ is other than hydrogen, except that when R⁴ is alkoxy, R³ is other than fluoro, when M is nitrogen, R⁴ is other than methyl, and when R⁴ is methyl, one of R², R³, R⁵ and R⁶ is other than hydrogen; and
including the diastereomers, enantiomers, racemates, tautomers, salts, esters, amides and prodrugs thereof.
